(19) United States
(12) Patent Application Publication
(10) Pub. No.: US 2011/0112288 A1
(43) Pub. Date: May 12, 2011

(54) Metal Porphyrin Catalyzed Olefin Aziridination with Sulfonyl Azides

(75) Inventors: X. Peter Zhang, Tampa, FL (US); Joshua V. Ruppel, Lutz, FL (US)

(73) Assignee: UNIVERSITY OF SOUTH FLORIDA, Tampa, FL (US)

(21) Appl. No.: 12/935,309

(22) PCT Filed: Mar. 31, 2009
(86) PCT No.: PCT/US09/38968

§ 371 Date: Dec. 27, 2010

Related U.S. Application Data
(60) Provisional application No. 61/041,206, filed on Mar. 31, 2008.

(51) Int. Cl. C07F 15/06; C07D 203/24

(52) U.S. Cl. 540/145; 548/965

(57) ABSTRACT

Cobalt(II) complex of P1 [Co(P1)], a new porphyrin that was designed on the basis of potential hydrogen bonding interactions in the metal-nitrene intermediate, is a highly active catalyst for olefin aziridination with azides. The [Co(P1)]-based system can be effectively employed for different combinations of aromatic olefins and arysulfonyl azides, synthesizing various sulfonylated aziridines in excellent yields. Besides its mild catalytic conditions, the Co-catalyzed aziridination process enjoys several attributes associated with the relatively low cost of cobalt and widely accessible arylsulfonyl azides. Furthermore, it generates stable dinitrogen as the only by-product.

### STATEMENT OF GOVERNMENT RIGHTS

This invention was made with Government support under grant number NSF #0711024, awarded by the National Science Foundation, Division of Chemistry, and under grant number CRIF: MU-0443611, awarded by the National Science Foundation. The Government has certain rights in the invention.

### CROSS-REFERENCE TO RELATED APPLICATIONS

This application claims priority to U.S. Provisional Application Ser. No. 61/041,206, filed Mar. 31, 2008, which is incorporated herein by reference in its entirety.

### FIELD OF THE INVENTION

The present invention generally relates to a catalytic system for and the aziridination of olefins and, in one embodiment, the cobalt porphyrin catalyzed aziridination of aromatic olefins with arylsulfonyl azides.

### BACKGROUND

Metal-catalyzed olefin aziridination is a fundamentally and practically important chemical process that has received increasing research attention. (Muller et al., Chem. Rev. 2003, 103, 2905; Hu, X. E., Tetrahedron 2004, 60, 2701.) The resulting aziridines, the smallest nitrogen-containing heterocyclic compounds, are key elements in many biologically and pharmaceutically interesting compounds and serve as a class of versatile synthons for preparation of functionalized amines. Since the introduction of Phl=NTs as a nitrene source more than three decades ago, considerable progress has been made in metal-catalyzed olefin aziridination with PHl=NTs and related iminoiodane derivatives, including the notable recent developments with the use of their in situ variants. (Yamada et al., Chem. Lett. 1975, 361. For recent examples with Phl=NTs: Klotz et al., Chem. Commun. 2007, 2063; Zdilla et al., J. Am. Chem. Soc. 2006, 128, 16971; Cui et al., J. Am. Chem. Soc. 2003, 125, 16202. For recent in situ variants, see: Esteoule et al., Synthesis 2007, 1251; Guthikonda et al., Tetrahedron 2006, 62,11331; Li et al., J. Org. Chem. 2006, 71, 5876; Xu et al., Org. Lett. 2008, 10, 1497. For other approaches, see: Antilla et al., J. Am. Chem. Soc. 1999, 121, 5099; Williams et al., J. Am. Chem. Soc. 2004, 126, 1612; Vyas et al., Org. Lett. 2004, 6, 1907; Catino et al., Org. Lett. 2005, 7, 2787.) Despite these advances, the search for alternative nitrene sources is warranted as the use of Phl=NTs has met with several difficulties. Besides its short shelf life and poor solubility in common solvents, aziridination with Phl=NTs generates a stoichiometric amount of Phl as a by-product. In view of the similarity to diazo reagents for carbene transfer processes, azides should have the potential to serve as a general class of nitrene sources for metal-mediated nitrene transfer reactions, including aziridination. In addition to their wide availability and ease of synthesis, azide-based nitrene transfers would generate chemically stable and environmentally benign nitrogen gas as the only by-product. Despite these attributes, only a few catalytic systems have been developed that can effectively catalyze the decomposition of azides for aziridination. (Scriven et al., Chem. Rev. 1988, 88, 297; Brase et al., Angew. Chem., Int. Ed. 2005, 44, 5188; Kwart et al., J. Am. Chem. Soc. 1967, 89, 1951; Li et al., J. Am. Chem. Soc. 1995, 117, 5889; Cenini et al., Coord. Chem. Rev. 2006, 250, 1234; Piangiolino et al., Eur. J. Org. Chem. 2007, 743; Katsuki, T. Chem. Lett. 2005, 1304; Kawabata et al., Chem. Asian J. 2007, 2, 248. For a Co-catalyzed hydroazidation of olefins, see: Waser et al., J. Am. Chem. Soc. 2005, 127, 8294. For a Bronsted acid-promoted process, see: Mahoney et al., J. Am. Chem. Soc. 2005, 127, 1354.)

### SUMMARY OF THE INVENTION

Among the various aspects of the present invention, therefore, is a process for the aziridination of olefins, and a cobalt catalyst for olefin aziridination.

Briefly, therefore, one aspect of the present invention is a process for the aziridination of an olefin, the process comprising treating the olefin with a sulfonyl azide in the presence of a metal porphyrin complex.

Another aspect of the present invention is a process for the aziridination of an olefin, the process comprising treating the olefin with a sulfonyl azide in the presence of a metal porphyrin complex, wherein the olefin corresponds to Formula 1, the sulfonyl azide corresponds to Formula A:

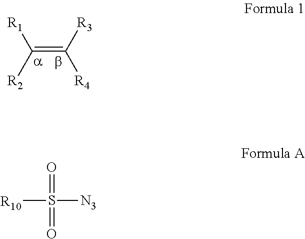

and R₁, R₂, R₃, R₄ and R₁₀ are independently hydrogen, hydrocarbyl, substituted hydrocarbyl, heterocyclo or EWG (electron-withdrawing group).

Another aspect of the present invention is a process for the aziridination of an olefin, the process comprising treating the olefin with a sulfonyl azide in the presence of a metal porphyrin complex, wherein the olefin corresponds to Formula 5, the sulfonyl azide corresponds to Formula B:

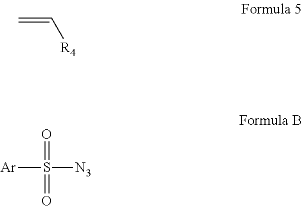

and R₄ and Ar are independently aryl.

Another aspect of the present invention is a cobalt porphyrin catalyst corresponding to the formula:

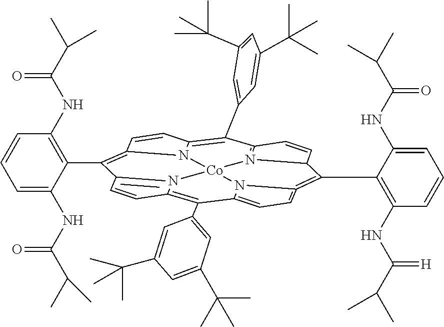

Other aspects of the invention will be, in part, apparent, and, in part, pointed out hereinafter.

### DETAILED DESCRIPTION OF THE PREFERRED EMBODIMENTS

In accordance with certain aspects of the present invention, a process and catalysts are provided for olefin aziridination with azides. In one aspect, a cobalt porphyrin complex is used as the catalyst. In another aspect, any of a wide range of olefins are aziridinated using any of a wide range of azides. In a preferred embodiment, an aromatic olefin is aziridinated with an arylsulfonylazide.

Olefins

In general, the olefin, also referred to herein as an alkene, may be any of a wide range of olefins. In one embodiment, the alkene is a terminal alkene. For example, the alkene may be a monosubstituted terminal alkene or a disubstituted terminal alkene. In another embodiment, the alkene is an internal alkene. For example, the alkene may be a disubstituted, trisubstituted or tetrasubstituted internal alkene. If disubstituted, the internal alkene may have the cis or trans configuration. In one embodiment, the olefin is an aromatic, monosubstituted terminal alkene.

In one embodiment, the olefin corresponds to Formula 1:

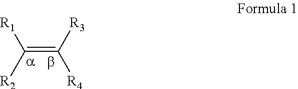

wherein R₁ and R₂ are substituents of the a-carbon of the ethylenic bond (also referred to as an olefinic bond), and R₃ and R₄ are substituents of the 8-carbon of the ethylenic bond. R₁, R₂, R₃, and R₄ are independently hydrogen, hydrocarbyl, substituted hydrocarbyl, heterocyclo or EWG (electron-withdrawing group). In one embodiment, R₁ is hydrogen. In another embodiment, R₁ is alkyl, substituted alkyl, or aryl. In one embodiment, R₂ is hydrogen. In another embodiment, R₂ is alkyl, substituted alkyl, or aryl. In one embodiment, R₃ is hydrogen. In another embodiment, R₃ is alkyl, substituted alkyl, or aryl. In one embodiment, R₄ is hydrogen. In another embodiment, R₄ is alkyl, substituted alkyl, or aryl. In one embodiment, two of R₁, R₂, R₃ and R₄ are hydrogen. In another embodiment, three of R₁, R₂, R₃, and R₄ are hydrogen. In one embodiment, R₁, R₂ and the α-carbon, or R₃, R₄ and the β-carbon, form a carbocyclic or heterocyclic ring. In another embodiment, R₁, R₃, the α-carbon, and the β-carbon, or R₂, R₄, the α-carbon, and the β-carbon form a carbocyclic or heterocyclic ring. In another embodiment, R₁, R₄, the a-carbon, and the β-carbon, or R₂, R₃, the α-carbon, and the β-carbon form a carbocyclic or heterocyclic ring. In one preferred embodiment, at least one of R₁, R₂, R₃, and R₄ is alkyl, aryl, substituted phenyl, —CN, —C(O)R₂₂, or —C(O)OR₂₂ wherein R₂₂ is hydrogen, optionally substituted alkyl, optionally substituted alkenyl, optionally substituted alkynyl, or optionally substituted aryl. In another presently preferred embodiment, one of R₁, R₂, R₃, and R₄ is aryl and the others are hydrogen; for example, in this embodiment, R₁, R₂, and R₃ may be hydrogen and R₄ is aryl, optionally substituted with any of the substituents identified elsewhere herein in connection with the substituted hydrocarbyl substituents.

When the olefin corresponds to Formula 1 and one of R₁, R₂, R₃, and R₄ is hydrogen, e.g., R₂ is hydrogen, the olefin corresponds to Formula 2:

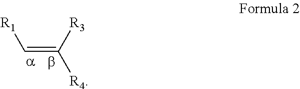

wherein R₁ is a substituent of the a-carbon of the ethylenic bond, R₃ and R₄ are substituents of the β-carbon of the ethylenic bond, and R₁, R₃, and R₄ are independently hydrogen, hydrocarbyl, substituted hydrocarbyl, or heterocyclo. In one embodiment, R₁ is hydrogen and the olefin is a mono or disubstituted terminal alkene. In another embodiment, R₁ is alkyl, substituted alkyl or aryl and the olefin is a disubstituted or a trisubstituted internal alkene. In one embodiment, R₃ is hydrogen. In another embodiment, R₃ is alkyl, substituted alkyl or aryl. In one embodiment, R₄ is hydrogen. In another embodiment, R₄ is alkyl, substituted alkyl or aryl. In one embodiment, two of R₁, R₃ and R₄ are hydrogen. In one embodiment, R₃, R₄ and the β-carbon form a carbocyclic or heterocyclic ring. In another embodiment, R₁, R₃, the α-carbon, and the β-carbon form a carbocyclic or heterocyclic ring. In another embodiment, R₁, R₄, the α-carbon, and the β-carbon form a carbocyclic or heterocyclic ring. In one preferred embodiment, at least one of R₁, R₃, and R₄ is alkyl, phenyl, substituted phenyl, —CN, —C(O)R₂₂, or —C(O)OR₂₂ wherein R₂₂ is optionally substituted alkyl, optionally substituted alkenyl, optionally substituted alkynyl, or optionally substituted aryl.

When the olefin corresponds to Formula 1, R₂ is hydrogen, and one of R₃ and R₄ is hydrogen, the olefin corresponds to Formula 3-cis or Formula 3-trans:

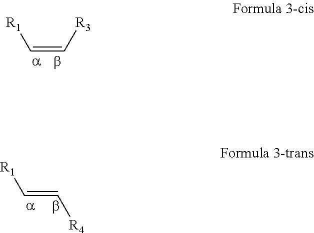

wherein R₁ is a substituent of the a-carbon of the ethylenic bond, and R₃ and R₄ are substituents of the β-carbon of the ethylenic bond. R₁, R₃ and R₄ are independently hydrogen, hydrocarbyl, substituted hydrocarbyl, or heterocyclo. In one embodiment, R₁ is hydrogen. In another embodiment, R₁ is alkyl, substituted alkyl or aryl. In one embodiment, R₃ is hydrogen. In another embodiment, R₃ is alkyl, substituted alkyl or aryl. In one embodiment, R₄ is hydrogen. In another embodiment, R₄ is alkyl, substituted alkyl or aryl. In one embodiment, R₁, R₃, the α-carbon, and the β-carbon form a carbocyclic or heterocyclic ring. In another embodiment, R₁, R₄, the α-carbon, and the —-carbon form a carbocyclic or heterocyclic ring. In one preferred embodiment, at least one of R₁, R₃, and R₄ is alkyl, alkenyl, heterocyclo, phenyl, substituted phenyl, —CN, —C(O)R₂₂, or —C(O)OR₂₂ wherein R₂₂ is optionally substituted alkyl, optionally substituted alkenyl, optionally substituted alkynyl, or optionally substituted aryl.

When the olefin corresponds to Formula 1 and two of the substituents on the same ethylenic carbon, e.g., R₁ and R₂, are each hydrogen, the olefin is a terminal alkene, corresponding to Formula 4:

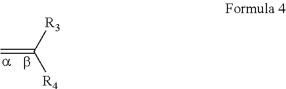

wherein R₃ and R₄ are substituents of the β-carbon of the ethylenic bond, and are independently hydrogen, hydrocarbyl, substituted hydrocarbyl, or heterocyclo. In one embodiment, R₃ is hydrogen. In another embodiment, R₃ is alkyl, substituted alkyl or aryl. In one embodiment, R₄ is hydrogen. In another embodiment, R₄ is alkyl, substituted alkyl or aryl. In one embodiment, R₃, R₄, and the β-carbon form a carbocyclic or heterocyclic ring. In one preferred embodiment, at least one of R₃ and R₄ is alkyl, alkenyl, heterocyclo, phenyl, substituted phenyl, —CN, —C(O)R₂₂, or —C(O)OR₂₂ wherein R₂₂ is optionally substituted alkyl, optionally substituted alkenyl, optionally substituted alkynyl, or optionally substituted aryl.

When the olefin corresponds to Formula 1 and three of R₁, R₂, R₃, and R₄ are hydrogen, e.g., R₁, R₂, and R₃ are hydrogen, the olefin is a terminal olefin corresponding to Formula 5:

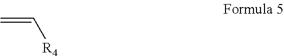

wherein R₄ is hydrogen, hydrocarbyl, substituted hydrocarbyl, or heterocyclo. In one embodiment, R₄ is alkyl, substituted alkyl or aryl. In another embodiment, R₄ is aryl. For example, R₄ may be phenyl, substituted phenyl, naphthyl, or substituted naphthyl. By way of further example, in one embodiment, R₄ is preferably phenyl or naphthyl, optionally substituted with alkyl, heterosubstituted alkyl, or a hetero atom containing substituent selected from the group consisting of halogen, heterocyclo, alkoxy, alkenoxy, alkynoxy, aryloxy, hydroxyl, protected hydroxy, keto, acyl, acyloxy, nitro, amino, amido, nitro, cyano, thiol, ketals, acetals, esters and ethers. In a further preferred embodiment, R₄ is phenyl, tolyl (CH₃C₆H₄—), tert-butyl phenyl, chlorophenyl, bromophenyl, fluorophenyl, trifluoromethyl phenyl, or naphthyl.

In a preferred embodiment, the olefin is an aromatic olefin corresponding to the following Formula 6:

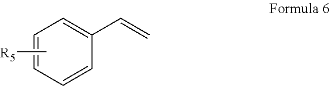

wherein R₅ is hydrogen, hydrocarbyl, substituted hydrocarbyl, heterocyclo, halo, alkoxy, alkenoxy, alkynoxy, aryloxy, hydroxy, keto, acyl, acyloxy, nitro, amino, amido, cyano, or thiol. In one embodiment, R₅ is hydrogen. In another embodiment, R₅ is alkyl. In another embodiment, R₅ is alkyl substituted with a substituent selected from the group consisting of halogen, heterocyclo, alkoxy, alkenoxy, alkynoxy, aryloxy, hydroxy, keto, acyl, acyloxy, nitro, amino, amido, nitro, cyano, thiol, ketals, acetals, esters and ethers. In one such embodiment, R₅ is a halogen. In one preferred embodiment, R₅ is methyl, butyl, chloro, bromo, fluoro, trifluoromethyl, alkoxy, hydroxy, keto, acyl, acyloxy, amino, amido, nitro, cyano, or thiol. In one preferred embodiment, R₅ is methyl, butyl, chloro, bromo, fluoro, trifluoromethyl, alkoxy, hydroxy, amino, amido, nitro, or cyano. In one preferred embodiment, R₅ is methyl, tert-butyl, chlorine, bromine, fluorine, or trifluoromethyl.

Azides

In general, the olefin is aziridinated with a nitrene source. Preferably, the nitrene precursor is an azide reagent (also sometimes referred to herein as an azide compound) wherein the nitrene is generated by the removal of N₂ as nitrogen gas from the solution.

In one embodiment, the nitrene source is a sulfonyl azide corresponding to the following Formula A:

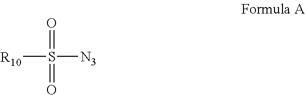

wherein R₁₀ is hydrogen, hydrocarbyl, substituted hydrocarbyl, or heterocyclo. In one embodiment, R₁₀ is alkyl, alkenyl, alkynyl, aryl, alkyl, substituted alkyl, substituted alkenyl, substituted alkynyl, or substituted aryl.

In one presently preferred embodiment, R₁₀ is aryl. For example, in this embodiment R₁₀ may be phenyl or substituted phenyl. By way of further example, the phenyl may be alkyl substituted (e.g., tolyl) or heterosubstituted. If heterosubstituted, the phenyl moiety is preferably substituted with a halogen, heterocyclo, alkoxy, alkenoxy, alkynoxy, aryloxy, hydroxy, acyl, acyloxy, nitro, amino, amido, cyano, or thiol moiety. For example, in one embodiment R₁₀ is phenyl, naphthyl, tolyl, methoxyphenyl, ethanoylamine phenyl (CH₃C(O)NHC₆H₄—) or other amido substituted phenyl, cyanophenyl, or nitrophenyl.

In a preferred embodiment, the nitrene source is an arylsulfonyl azide corresponding to the following Formula B:

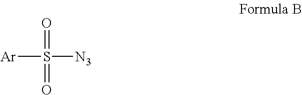

wherein Ar is aryl, including optionally substituted aryl. In a preferred embodiment, Ar is phenyl, tolyl, methoxyphenyl, ethanoylamine phenyl (CH₃C(O)NHC₆H₄—) or other amido substituted phenyl, cyanophenyl, nitrophenyl, or naphthyl.

Metal Porphyrin Complex

An aspect of the present invention is a process for the aziridination of olefins in the presence of a catalyst. In an embodiment, the catalyst is a metal porphyrin complex. In one embodiment, the metal of the metal porphyrin complex is a transition metal. Thus, for example, the metal, M, may be any of the 30 metals in the 3d, 4d, and 5d transition metal series of the Periodic Table of the Elements, including the 3d series that includes Sc, Ti, V, Cr, Mn, Fe, Co, Ni, Cu, and Zn; the 4d series that includes Y, Zr, Nb, Mo, Tc, Ru, Rh, Pd, Ag and Cd; and the 5d series that includes Lu, Hf, Ta, W, Re, Os, Ir, Pt, Au and Hg. In some embodiments, M is a transition metal from the 3d series. In some embodiments, M is selected from the group consisting of Co, Zn, Fe, Ru, Mn, and Ni. In some embodiments, M is selected from the group consisting of Co, Fe, and Ru. In some embodiments, M is Co.

The porphyrin with which the metal is complexed may be any of a wide range of porphyrins known in the art. Exemplary porphyrins are described in U.S. Patent Publication Nos. 2005/0124596 and 2006/0030718 and U.S. Pat. No. 6,951,935 (each of which is incorporated herein by reference, in its entirety). Exemplary porphyrins are also described in Chen et al., Bromoporphyrins as Versatile Synthons for Modular Construction of Chiral Porphyrins: Cobalt-Catalyzed Highly Enantioselective and Diastereoselective Cyclopropanation (J. Am. Chem. Soc. 2004), which is incorporated herein by reference in its entirety.

In a preferred embodiment, the porphyrin is complexed with cobalt. The porphyrin with which cobalt is complexed may be any of a wide range of porphyrins known in the art. Exemplary porphyrins are described in U.S. Patent Publication Nos. 2005/0124596 and 2006/0030718 and U.S. Pat. No. 6,951,935 (each of which is incorporated herein by reference, in its entirety). Exemplary porphyrins are also described in Chen et al., Bromoporphyrins as Versatile Synthons for Modular Construction of Chiral Porphyrins: Cobalt-Catalyzed Highly Enantioselective and Diastereoselective Cyclopropanation (J. Am. Chem. Soc. 2004), which is incorporated herein by reference in its entirety.

Generally, a preferred cobalt for aziridinating olefins is a cobalt porphyrin complex. In one embodiment, the cobalt porphyrin complex is a cobalt (II) porphyrin complex. In one particularly preferred embodiment, the cobalt porphyrin complex is a D₂-symmetric chiral porphyrin complex corresponding to the following structure:

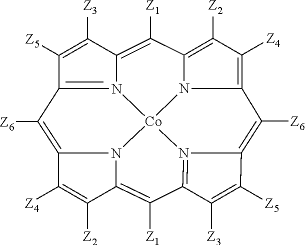

wherein each Z₁, Z₂, Z₃, Z₄, Z₅ and Z₆ are each independently selected from the group consisting of X, H, alkyl, substituted alkyls, arylalkyls, aryls and substituted aryls; and X is selected from the group consisting of halogen, triflouromethanesulfonate (OTf), haloaryl and haloalkyl. In a preferred embodiment, Z₂, Z₃, Z₄ and Z₅ are hydrogen, Z₁ is a substituted phenyl, and Z₆ is substituted phenyl, and Z₁ and Z₆ are different. In one particularly preferred embodiment, Z₂, Z₃, Z₄ and Z₅ are hydrogen, Z₁ is substituted phenyl, and Z₆ is substituted phenyl and Z₁ and Z₆ are different and the porphyrin is a chiral porphyrin. In one even further preferred embodiment, Z₂, Z₃, Z₄ and Z₅ are hydrogen, Z₁ is substituted phenyl, and Z₆ is substituted phenyl and Z₁ and Z₆ are different and the porphyrin has D₂-symmetry. In one embodiment, Z₂, Z₃, Z₄ and Z₅are hydrogen, Z₁ is 3,5-di(tert-butyl)phenyl, and Z₆ is 2,6-di(isobutylamine)phenyl.

Exemplary cobalt (II) porphyrins include the following:

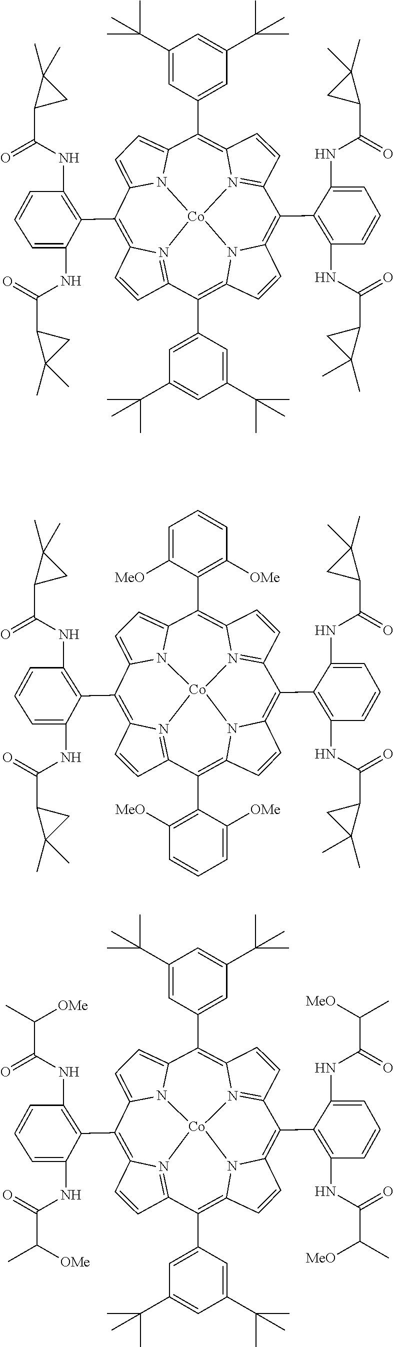

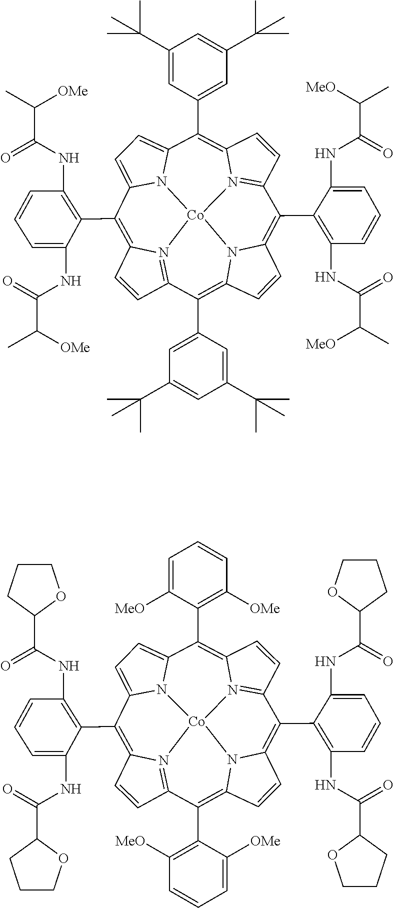

Aziridination Reactions

accordance with one embodiment of the present invention, an alkene is converted to an aziridine as illustrated in Reaction Scheme A:

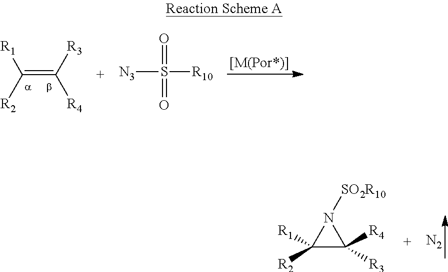

wherein [M(Por*)] is a metal porphyrin complex, R₁, R₂, R₃ and R₄ are independently hydrogen, hydrocarbyl, substituted hydrocarbyl, heterocyclo or EWG (electron-withdrawing group), and R₁₀ is hydrogen, hydrocarbyl, substituted hydrocarbyl, or heterocyclo. In one embodiment, the metal porphyrin complex is a cobalt porphyrin complex. In one embodiment, R₁₀ is alkyl, alkenyl, alkynyl, phenyl, alkyl, substituted alkyl, or heterosubstituted phenyl. In another embodiment, R₁₀ is phenyl, tolyl, methoxyphenyl, ethanoylamine phenyl, cyanophenyl, nitrophenyl, or naphthyl. In one embodiment, R₁ is hydrogen. In another embodiment, R₁ is alkyl, substituted alkyl or aryl. In one embodiment, R₂ is hydrogen. In another embodiment, R₂ is alkyl, substituted alkyl or aryl. In one embodiment, R₃ is hydrogen. In another embodiment, R₃ is alkyl, substituted alkyl or aryl. In one embodiment, R₄ is hydrogen. In another embodiment, R₄ is alkyl, substituted alkyl or aryl. In one embodiment, at least one of R₁, R₂, R₃ and R₄ is hydrogen and the other three are alkyl, substituted alkyl or aryl. In one embodiment, at least two of R₁, R₂, R₃ and R₄ are hydrogen and the other two are alkyl, substituted alkyl or aryl. In another embodiment, at least three of R₁, R₂, R₃ and R₄ are hydrogen and the other one is alkyl, substituted alkyl or aryl. In one embodiment, R₁, R₂ and the aziridine ring carbon to which they are bonded, or R₃, R₄ and the aziridine ring carbon to which they are bonded, form a carbocyclic or heterocyclic ring. In another embodiment, R₁, R₃, and the aziridine ring carbons to which R₁ and R₃ are bonded, or R₂, R₄, and the aziridine ring carbons to which R₂ and R₄ are bonded, form a carbocyclic or heterocyclic ring. In another embodiment, R₁, R₄, and the aziridine ring carbons to which R₁ and R₄ are bonded, or R₂, R₃, and the aziridine ring carbons to which R₂ and R₃ are bonded, form a carbocyclic or heterocyclic ring.

In one embodiment, the aziridination reaction proceeds as illustrated in Reaction Scheme B:

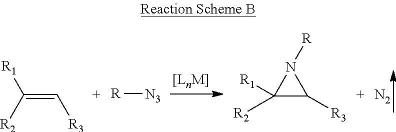

wherein R₁, R₂, and R₃ are as previously described in connection with the olefin, R is a sulfonyl, and [LₙM] is a metal catalyst.

In one embodiment, an alkene is aziridinated as illustrated in Reaction Scheme B-1, B-2, B-3, or B-4.

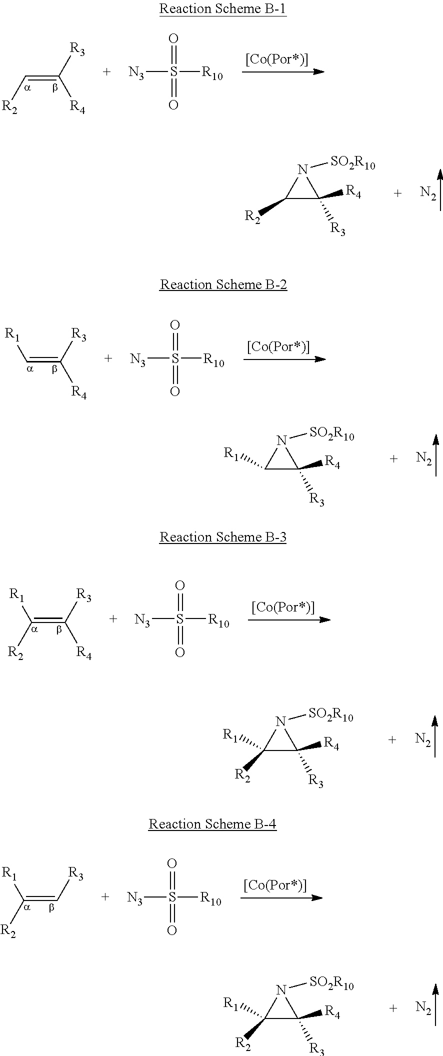

wherein [Co(Por*)] is a cobalt porphyrin complex, R₁, R₂, R₃ and R₄ are independently hydrogen, hydrocarbyl, substituted hydrocarbyl, or heterocyclo, and R₁₀ is hydrogen, hydrocarbyl, substituted hydrocarbyl, or heterocyclo. In one embodiment, R₁₀ is alkyl, alkenyl, alkynyl, phenyl, alkyl, substituted alkyl, or heterosubstituted phenyl. In another embodiment, R₁₀ is phenyl, tolyl, methoxyphenyl, ethanoylamine phenyl, cyanophenyl, nitrophenyl, or naphthyl. In one embodiment, R₁ is hydrogen. In another embodiment, R₁ is alkyl, substituted alkyl or aryl. In one embodiment, R₂ is hydrogen. In another embodiment, R₂ is alkyl, substituted alkyl or aryl. In one embodiment, R₃ is hydrogen. In another embodiment, R₃ is alkyl, substituted alkyl or aryl. In one embodiment, R₄ is hydrogen. In another embodiment, R₄ is alkyl, substituted alkyl or aryl. In one embodiment, at least one of R₁, R₂, R₃ and R₄ is hydrogen and the other two are independently alkyl, substituted alkyl or aryl. In one embodiment, at least two of R₁, R₂, R₃ and R₄ are hydrogen and the other one is alkyl, substituted alkyl or aryl. In one embodiment, R₁, R₂ and the aziridine ring carbon to which they are bonded, or R₃, R₄ and the aziridine ring carbon to which they are bonded, form a carbocyclic or heterocyclic ring. In another embodiment, R₁, R₃, and the aziridine ring carbons to which R₁ and R₃ are bonded, or R₂, R₄, and the aziridine ring carbons to which R₂ and R₄ are bonded, form a carbocyclic or heterocyclic ring. In another embodiment, R₁, R₄, and the aziridine ring carbons to which R₁ and R₄ are bonded, or R₂, R₃, and the aziridine ring carbons to which R₂ and R₃ are bonded, form a carbocyclic or heterocyclic ring.

In one preferred embodiment, an alkene is aziridinated as illustrated in Reaction Scheme C-1:

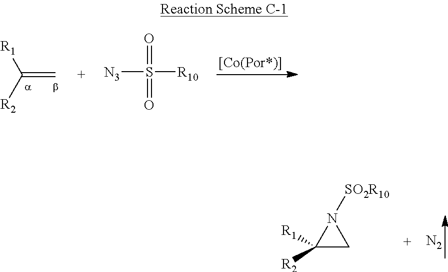

wherein [Co(Por*)] is a cobalt porphyrin complex, R₁ and R₂ are independently hydrogen, hydrocarbyl, substituted hydrocarbyl, or heterocyclo, and R₁₀ is hydrogen, hydrocarbyl, substituted hydrocarbyl, or heterocyclo. In one embodiment, R₁₀ is alkyl, alkenyl, alkynyl, phenyl, alkyl, substituted alkyl, or heterosubstituted phenyl. In another embodiment, R₁₀ is phenyl, tolyl, methoxyphenyl, ethanoylamine phenyl (or other amido substituted phenyl), cyanophenyl, nitrophenyl, or naphthyl. In one embodiment, R₁ is hydrogen. In another embodiment, R₁ is alkyl, substituted alkyl or aryl. In one embodiment, R₂ is hydrogen. In another embodiment, R₂ is alkyl, substituted alkyl or aryl. In one embodiment, one of R₁ and R₂ is hydrogen and the other one is alkyl, substituted alkyl or aryl. In one embodiment, R₁, R₂ and the aziridine ring carbons to which they are bonded form a carbocyclic or heterocyclic ring.

In another preferred embodiment, an alkene is converted to an aziridine as illustrated in Reaction Scheme C-2-trans:

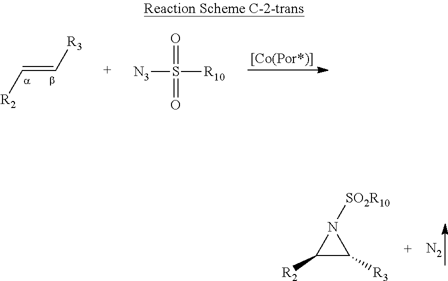

wherein [Co(Por*)] is a cobalt porphyrin complex, R₂ and R₃ are independently hydrogen, hydrocarbyl, substituted hydrocarbyl, or heterocyclo, and R₁₀ is hydrogen, hydrocarbyl, substituted hydrocarbyl, or heterocyclo. In one embodiment, R₁₀ is alkyl, alkenyl, alkynyl, phenyl, alkyl, substituted alkyl, or heterosubstituted phenyl. In another embodiment, R₁₀ is phenyl, tolyl, methoxyphenyl, ethanoylamine phenyl, cyanophenyl, nitrophenyl, or naphthyl. In one embodiment, R₂ is hydrogen. In another embodiment, R₂ is alkyl, substituted alkyl or aryl. In one embodiment, R₃ is hydrogen. In another embodiment, R₃ is alkyl, substituted alkyl or aryl. In one embodiment, one of R₂ and R₃ is hydrogen and the other one is alkyl, substituted alkyl or aryl. In one embodiment, R₂, R₃, and the aziridine ring carbons to which they are bonded form a carbocyclic or heterocyclic ring.

In another preferred embodiment, an alkene is converted to an aziridine as illustrated in Reaction Scheme C-2-cis:

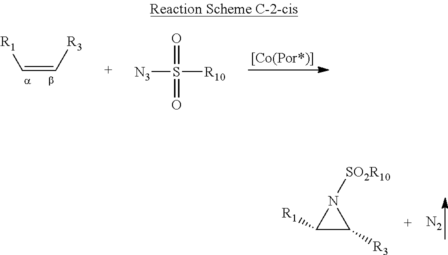

wherein [Co(Por*)] is a cobalt porphyrin complex, R₁ and R₃ are independently hydrogen, hydrocarbyl, substituted hydrocarbyl, or heterocyclo, and R₁₀ is hydrogen, hydrocarbyl, substituted hydrocarbyl, or heterocyclo. In one embodiment, R₁₀ is alkyl, alkenyl, alkynyl, phenyl, alkyl, substituted alkyl, or heterosubstituted phenyl. In another embodiment, R₁₀ is phenyl, tolyl, methoxyphenyl, ethanoylamine phenyl, cyanophenyl, nitrophenyl, or naphthyl. In one embodiment, R₁ is hydrogen. In another embodiment, R₁ is alkyl, substituted alkyl or aryl. In one embodiment, R₃ is hydrogen. In another embodiment, R₃ is alkyl, substituted alkyl or aryl. In one embodiment, one of R₁ and R₃ is hydrogen and the other one is alkyl, substituted alkyl or aryl. In one embodiment, R₁, R₃, and the aziridine ring carbons to which R₁ and R₃ are bonded form a carbocyclic or heterocyclic ring.

In another preferred embodiment, an alkene is converted to an aziridine as illustrated in Reaction Scheme D:

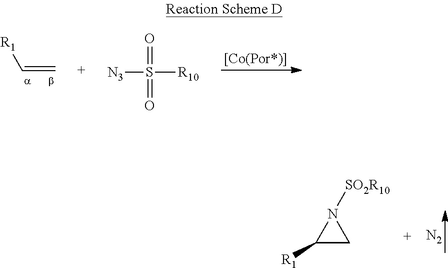

wherein [Co(Por*)] is a cobalt porphyrin complex, R₁ is hydrogen, hydrocarbyl, substituted hydrocarbyl, or heterocyclo, and R₁₀ is hydrogen, hydrocarbyl, substituted hydrocarbyl, or heterocyclo. In one embodiment, R₁₀ is alkyl, alkenyl, alkynyl, phenyl, alkyl, substituted alkyl, or heterosubstituted phenyl. In one presently preferred embodiment, R₁₀ is aryl. In another embodiment, R₁₀ is phenyl, tolyl, methoxyphenyl, ethanoylamine phenyl, cyanophenyl, nitrophenyl, or naphthyl. In one embodiment, R₁ is alkyl substituted alkyl, or aryl. In one presently preferred embodiment, R₁ is aryl and R₁₀ is aryl.

In a preferred embodiment, an olefin is aziridinated in the presence of an azide and a cobalt porphyrin catalyst as illustrated in Reaction Scheme 1:

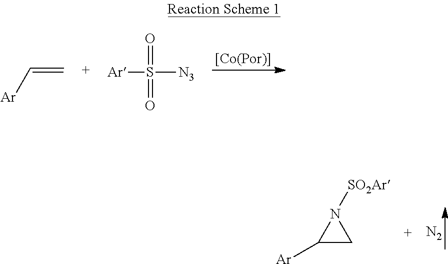

wherein Ar and Ar′ are independently aryl, that is, Ar and Ar′ are each aryl and are the same or are different, and wherein [Co(Por)] is a cobalt porphyrin complex catalyst.

In another preferred embodiment, an olefin is converted to an aziridine in the presence of an azide and a cobalt porphyrin catalyst as illustrated Reaction Scheme 2:

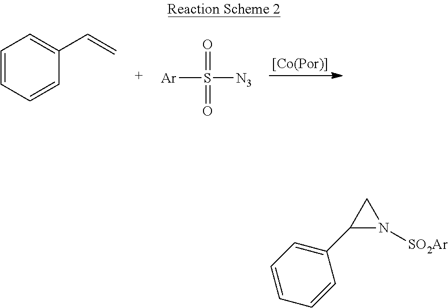

wherein Ar is aryl, and [Co(Por)] is a cobalt porphyrin complex catalyst. In one presently preferred embodiment, Ar is phenyl, tolyl, methoxyphenyl, ethanoylamine phenyl, cyanophenyl, nitrophenyl, or naphthyl.

In a further preferred embodiment, the cobalt catalyst aziridinates an olefin in the presence of an arylsulfonyl azide as illustrated in the following Reaction Scheme 3:

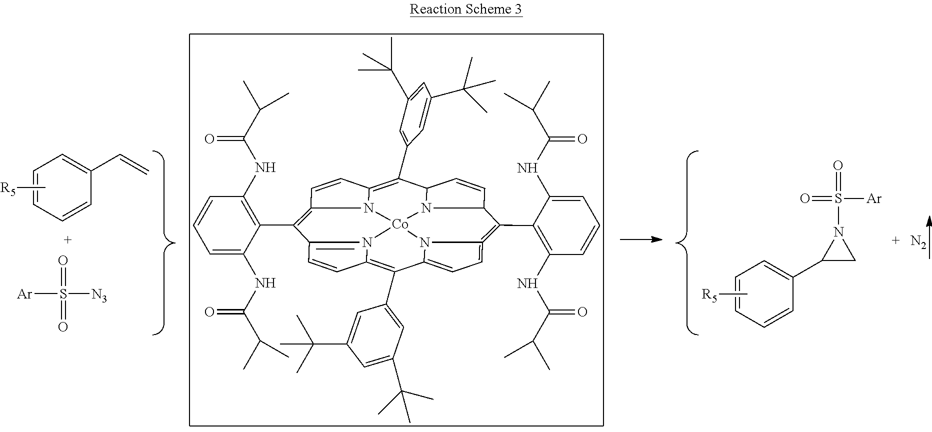

wherein R₅ is hydrogen, hydrocarbyl, substituted hydrocarbyl, heterocyclo, halo, alkoxy, alkenoxy, alkynoxy, aryloxy, hydroxy, acyl, acyloxy, nitro, amino, amido, cyano, or thiol. In one embodiment, R₅ is hydrogen. In another embodiment, R₅ is alkyl. In another embodiment, R₅ is alkyl substituted with a substituent selected from the group consisting of halogen, heterocyclo, alkoxy, alkenoxy, alkynoxy, aryloxy, hydroxy, keto, acyl, acyloxy, nitro, amino, amido, nitro, cyano, thiol, ketals, acetals, esters and ethers. In one embodiment, R₅ is a halogen. In one preferred embodiment, R₅ is methyl, butyl, chloro, bromo, fluoro, trifluoromethyl, alkoxy, hydroxy, keto, acyl, acyloxy, amino, amido, nitro, cyano, or thiol. In one presently preferred embodiment, R₅ is methyl, butyl, chloro, bromo, fluoro, trifluoromethyl, alkoxy, hydroxy, amino, amido, nitro, or cyano. In another presently preferred embodiment, R₅ is methyl, tert-butyl, chlorine, bromine, fluorine, or trifluoromethyl. In a further presently preferred embodiment, R₅ is hydrogen, hydrocarbyl, substituted hydrocarbyl, heterocyclo, or halo, and Ar is aryl, including without limitation optionally substituted aryl. In one embodiment, R₅ is hydrogen. In another embodiment, R₅ is alkyl. In a further presently preferred embodiment, R₅ is alkyl, substituted alkyl, halogen, heterocyclo, alkoxy, alkenoxy, alkynoxy, aryloxy, hydroxyl, protected hydroxy, acyl, acyloxy, nitro, amino, amido, cyano, or thiol, and Ar is phenyl or phenyl substituted with halogen, heterocyclo, alkoxy, alkenoxy, alkynoxy, aryloxy, hydroxyl, protected hydroxy, acyl, acyloxy, nitro, amino, amido, cyano, or thiol. In a further embodiment, R₅ is methyl, tert-butyl, chlorine, bromine, fluorine, or trifluoromethyl and Ar is phenyl, tolyl, alkoxyphenyl, amidophenyl, cyanophenyl, nitrophenyl, or naphthyl.

Abbreviations and Definitions

The following definitions and methods are provided to better define the present invention and to guide those of ordinary skill in the art in the practice of the present invention. Unless otherwise noted, terms are to be understood according to conventional usage by those of ordinary skill in the relevant art.

The term “acyl,” as used herein alone or as part of another group, denotes the moiety formed by removal of the hydroxyl group from the group —COOH of an organic carboxylic acid, e.g., RC(O)—, wherein R is _{R}¹, R¹O—, R¹ R²N—, or R¹S—, R¹ is hydrocarbyl, heterosubstituted hydrocarbyl, or heterocyclo and R² is hydrogen, hydrocarbyl or substituted hydrocarbyl.

The term “acyloxy,” as used herein alone or as part of another group, denotes an acyl group as described above bonded through an oxygen linkage (—O—), e.g., RC(O)O— wherein R is as defined in connection with the term “acyl.”

Unless otherwise indicated, the alkenyl groups described herein are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

Unless otherwise indicated, the alkyl groups described herein are preferably lower alkyl containing from one to eight carbon atoms in the principal chain and up to 20 carbon atoms. They may be straight or branched chain or cyclic and include methyl, ethyl, propyl, isopropyl, butyl, hexyl, and the like. The substituted alkyl groups described herein may have, as substituents, any of the substituents identified as substituted hydrocarbyl substituents.

The term alkoxy or alkoxyl shall mean any univalent radical, RO⁻ where R is an alkyl group.

Unless otherwise indicated, the alkynyl groups described herein are preferably lower alkynyl containing from two to eight carbon atoms in the principal chain and up to 20 carbon atoms. They may be straight or branched chain and include ethynyl, propynyl, butynyl, isobutynyl, hexynyl, and the like.

The term “amido” as used herein alone or as part of another group, denotes the moiety formed by removal of a hydrogen from the nitrogen atom of an amide, e.g., R¹OC(O)N(R²)— wherein R¹ and R² are independently hydrogen, hydrocarbyl, heterosubstituted hydrocarbyl, or heterocyclo. Thus, for example, amidophenyl or amido substituted phenyl may be R¹OC(O)N(R²)C₆H₄— wherein R¹ and R² are independently hydrogen, hydrocarbyl, heterosubstituted hydrocarbyl, or heterocyclo.

The terms “aryl” or “ar” as used herein alone or as part of another group denote optionally substituted homocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. Phenyl and substituted phenyl are the more preferred aryl. The substituted aryl groups described herein may have, as substituents, any of the substituents identified as substituted hydrocarbyl substituents.

The term “azide” as used herein describes a compound with three linked nitrogen atoms, including without limitation the anion with the formula N₃⁻ and the functional group Rₓ-N₃, wherein Rₓ is any atom in the Periodic Table of the Elements.

The terms “EWG” and “electron withdrawing group” describes any substituent that draws electrons away from the ethylenic bond. Exemplary electron withdrawing groups include hydroxy, alkoxy, mercapto, halogens, carbonyls, sulfonyls, nitrile, quaternary amines, nitro, trihalomethyl, imine, amidine, oxime, thioketone, thioester, or thioamide. In one embodiment, the electron withdrawing group(s) is/are hydroxy, alkoxy, mercapto, halogen, carbonyl, sulfonyl, nitrile, quaternary amine, nitro, or trihalomethyl. In another embodiment, the electron withdrawing group(s) is/are halogen, carbonyl, nitrile, quaternary amine, nitro, or trihalomethyl. In another embodiment, the electron withdrawing group(s) is/are halogen, carbonyl, nitrile, nitro, or trihalomethyl. When the electron withdrawing group is alkoxy, it generally corresponds to the formula —OR where R is hydrocarbyl, substituted hydrocarbyl, or heterocyclo. When the electron withdrawing group is mercapto, it generally corresponds to the formula —SR where R is hydrogen, hydrocarbyl, substituted hydrocarbyl or heterocyclo. When the electron withdrawing group is a halogen atom, the electron withdrawing group may be fluoro, chloro, bromo, or iodo; typically, it will be fluoro or chloro. When the electron withdrawing group is a carbonyl, it may be an aldehyde (—C(O)H), ketone (—C(O)R), ester (—C(O)OR), acid (—C(O)OH), acid halide (—C(O)X), amide (—C(O)NRₐR_{b}), or anhydride (—C(O)OC(O)R) where R is hydrocarbyl, substituted hydrocarbyl or heterocyclo, Ra and Rb are independently hydrogen, hydrocarbyl, substituted hydrocarbyl or heterocyclo, and X is a halogen atom. When the electron withdrawing group is a sulfonyl, it may be an acid (—SO₃H) or a derivative thereof (—SO₂R) where R is hydrocarbyl, substituted hydrocarbyl or heterocyclo. When the electron withdrawing group is a quaternary amine, it generally corresponds to the formula —N⁺RₐR_{b}R_{c} where Rₐ, R_{b} and R_{c} are independently hydrogen, hydrocarbyl, substituted hydrocarbyl or heterocyclo. When the electron withdrawing group is a trihalomethyl, it is preferably trifluoromethyl or trichloromethyl. In each of the foregoing exemplary electron withdrawing groups containing the variable “X”, in one embodiment, X may be chloro or fluoro, preferably fluoro. In each of the foregoing exemplary electron withdrawing groups containing the variable “R”, R may be alkyl. In each of the foregoing exemplary electron withdrawing groups containing the variable “Rₐ” and “R_{b}”, Rₐ and R_{b} may independently be hydrogen or alkyl.

The terms “halogen” or “halo” as used herein alone or as part of another group refer to chlorine, bromine, fluorine, and iodine.

The term “heteroatom” shall mean atoms other than carbon and hydrogen.

The term “heteroaromatic” as used herein alone or as part of another group denote optionally substituted aromatic groups having at least one heteroatom in at least one ring, and preferably 5 or 6 atoms in each ring. The heteroaromatic group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heteroaromatics include furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxy, protected hydroxy, acyl, acyloxy, alkoxy, alkenoxy, alkynoxy, aryloxy, halogen, amido, amino, nitro, cyano, thiol, ketals, acetals, esters and ethers.

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl, and alkynaryl. Unless otherwise indicated, these moieties preferably comprise 1 to 20 carbon atoms. The “substituted hydrocarbyl” moieties described herein are hydrocarbyl moieties which are substituted with at least one atom other than carbon, including moieties in which a carbon chain atom is substituted with a hetero atom such as nitrogen, oxygen, silicon, phosphorous, boron, sulfur, or a halogen atom. These substituents include halogen, heterocyclo, alkoxy, alkenoxy, alkynoxy, aryloxy, hydroxyl, protected hydroxy, keto, acyl, acyloxy, nitro, amino, amido, nitro, cyano, thiol, ketals, acetals, esters and ethers.

The term porphyrin refers to a compound comprising a fundamental skeleton of four pyrrole nuclei united through the a-positions by four methane groups to form the following macrocyclic structure:

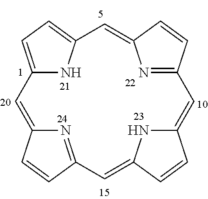

### EXAMPLES

We recently reported a Co-based system for catalytic aziridination with azide. (Gao et al., J. Org. Chem. 2006, 71, 6655.) It was shown that [Co(TPP)] can catalyze olefin aziridination with commercially available dipenylphosphoryl azide (DPPA) as a convenient new nitrene source, leading to the formation of N-phosphorylated aziridines. In an attempt to expand the catalytic process for other azides, it was found that [Co(TPP)] was ineffective for olefination aziridination with sulfonyl azides. For example, the desired aziridines 2a-c were obtained only in 11-24% yields from styrene when the common azides 1a-c were used (See Reaction Scheme 4). Changing the catalyst to Co(TDCIPP), which was shown to be effective for aziridination with bromamine-T, produced the desired product in less than 5% yield for each of the cases (Reaction Scheme 4); except unreacted azides and styrene, no other products were observed. (Gao et al., Org. Lett. 2005, 7, 3191.) As part of our efforts to develop new porphyrin ligands to enhance Co-based catalytic processes, herein we describe the design and . synthesis of a new porphyrin P1 based on potential hydrogen bonding interaction in the assumed metal-nitrene intermediate. The Co(II) complex of P1 [Co(P1)] was shown to be a highly active catalyst for aziridination of different aromatic olefins with various arylsulfonyl azides, forming the corresponding aziridines in excellent yields under mild conditions (Reaction Scheme 4). Careful control experiments showed that arylsulfonyl azides reported in this work were stable under the conditions used. But it should be noted that some of the azide compounds may be explosive and should be handled with great care.

Reaction Scheme 4.
Co-Catalyzed Aziridination of Styrene with Azides.

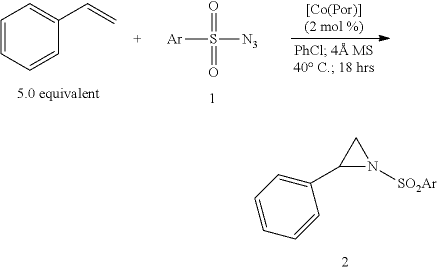

| ArSO₂N₃ (1) | [Co(TPP)] | [Co(TDClPP)] | [Co(P1)] |
|---|---|---|---|
| 4-Me—C₆H₄SO₂N₃ (1a) | 2a: 18% | 2a: <5% | 2a: 94% |
| 4-MeO—C₆H₄SO₂N₃ (1b) | 2b: 24% | 2b: <5% | 2b: 88% |
| 4-MeC(O)NH—C₆H₄SO₂N₃ | 2c: 11% | 2c: <5% | 2c: 98% |
| (1c) | | | |

[Co(TPP)] 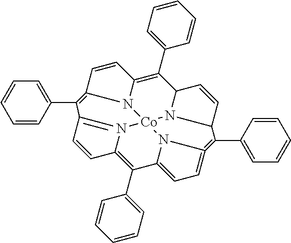
[Co(TDClPP)] 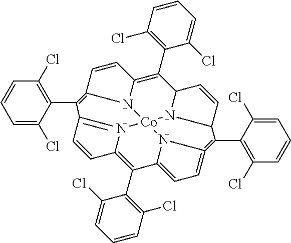
[Co(P1)]

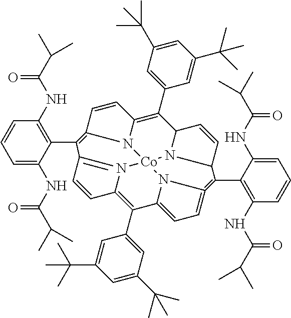

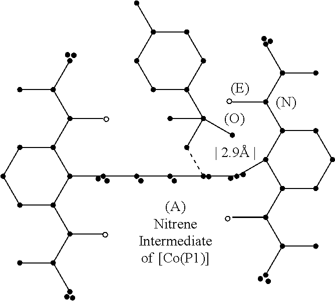

Similar to that proposed for other metal-based systems, the Co-catalyzed aziridination can be assumed to proceed via a mechanism involving a key electrophilic Co-nitrene intermediate. (Ruppel et al., Org. Lett. 2007, 9, 4889.) Accordingly, elements that can stabilize the formation of and enhance the electrophilicity of the nitrene intermediate should facilitate the catalytic cycle. Due to the existence of SO₂ group in sulfonyl azides, the D₂ₕ-symmetric porphyrin P1 containing amide functionalities at the ortho positions of meso-phenyl groups was designed to invoke a significant hydrogen bonding interaction between the S═O and N-H unit in the supposed nitrene intermediate of [Co(P1)]. (Simple computer modeling by molecular mechanics with Spartan 04 resulted in a minimized geometry with an O—N—H distance of 2.9 Å, suggesting a possibility of significant hydrogen bonding interaction. It should be noted that there is no experimental evidence for such interactions other than the modeling.) As a result of stabilization and activation of the nitrene intermediate of [Co(P1)] from the hydrogen bonding interaction, [Co(P1)] was expected to be a superior catalyst, in comparison with [Co(TPP)] and [Co(TDCIPP)], for aziridination with sulfonyl azides. (For an example of stabilization and activation of reactive intermediate by hydrogen bonding interactions, see: Lucas et al., J. Am. Chem. Soc. 2006, 128, 15476.)

[Co(P1)] was synthesized from its tetrabrominated precursor via a Pd-mediated quadruple amidation reaction with isobutylamide by following the previously established method. (Chen et al., J. Am. Chem. Soc. 2004, 126, 14718; Chen et al., J. Am. Chem. Soc. 2007, 129, 12074; Zhu et al., J. Am. Chem. Soc. 2008, 130, 5042.) [Co(P1)] was readily prepared from reaction of P1 with CoCl₂ in THF in the presence of 2,6-lutidine. Under the same conditions used for the aforementioned reactions by [Co(TPP)] and [Co(TDCIPP)], we were delighted to find that employment of [Co(P1)] resulted in a dramatic improvement of the catalytic aziridination (Reaction Scheme 4). The desired aziridines 2a, 2b, and 2c were obtained in 94%, 88%, and 98% isolated yields, respectively, supporting the hydrogenation bonding-guided catalyst design. ([Co(P1)] could effectively catalyze aziridination of styrene with Phl=NTs, forming the desired aziridines in 84% isolated yield.)

In addition to azides 1a, 1b, and 1c that contain -methyl, -methoxy, and -acetamide groups (Table 1, entries 1-3), [Co(P1)] could effectively activate a wide range of arylsulfonyl azides for aziridination (Table 1). For example, the use of arylsulfonyl azides having para-cyano (1d), para-nitro (1e), and ortho-nitro (1f) substituents afforded the corresponding aziridination products of styrene 2d-2f in excellent yields (Table 1, entries 4-6). Naphthalene-1-sulfonyl azide 1g was found to be an equally active nitrene source (Table 1, entry 7). Although the current [Co(P1)]-based catalytic system was ineffective for multiple substituted and aliphatic olefins, the [Co(P1)]-based catalytic aziridination system could be successfully applied to various combinations of arylsulfonyl azides and aromatic olefins (Table 2). For example, using azide le as a nitrene source, various styrene derivatives as well as 2-vinylnaphathene could be aziridinated in high to excellent yields (Table 2, entries 1-10). Similar results were obtained for azide 1c (Table 2, entries 11-14). While most of the reactions were carried out with 5 equiv. of olefin, the catalytic process could be operated with olefins as the limiting reagent as demonstrated with some selected examples, albeit in relatively lower yields (Table 2, entries 1, 2, 5, 6, and 8).

In summary, guided by hydrogen bonding interaction in the proposed intermediate, we designed and synthesized the new porphyrin P1 whose Co complex [Co(P1)] was shown to be a highly effective catalyst for aziridination of aromatic olefins with arylsulfonyl azides under mild conditions. Efforts are underway to expand the substrate scope to include non-aromatic olefins and to develop its asymmetric variants.

TABLE 1
[Co(P1]-Catalyzed Aziridination of Styrene with Azides ^{a}

| | | | yield |
|---|---|---|---|
| entry | azide | Aziridine | (%) ^{b} |
| 1 | 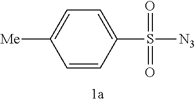 | 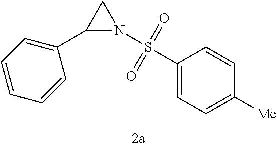 | 94 |
| 2 | 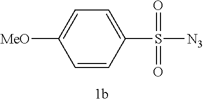 | 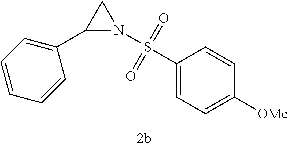 | 88 |
| 3 | 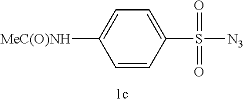 | 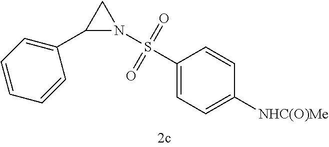 | 98 |
| 4 | 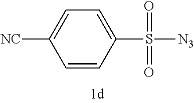 | 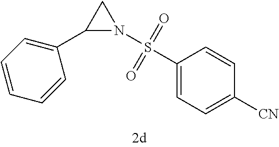 | 88 |
| 5 | 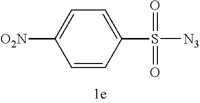 | 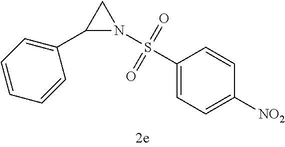 | 97 |
| 6 | 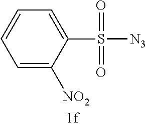 | 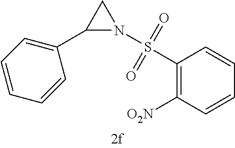 | 96 |
| 7 | 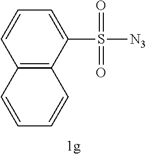 | 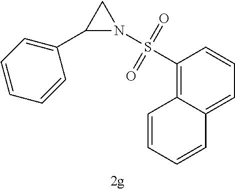 | 97 |

^{a} Reactions were carried out for 18 hours in chlorobenzene at 40° C. under N₂ in the presence of 4 Å molecular sieves using 2 mol % [Co(P1)];
Concentration: 0.20 mmol of azide/1 mL of chlorobenzene; Styrene: Azide = 5:1.
^{b} Isolated yields.

TABLE 2
Aziridination of Aromatic Olefins with Azides by [Co(P1)] ^{a}

| | | | | yield |
|---|---|---|---|---|
| entry | Azide | Olefin | aziridine | (%) ^{b} |
| 1 | 1e | 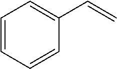 | 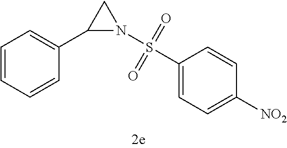 | 97 (90)^{c} |
| 2 | 1e | 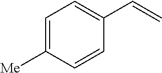 | 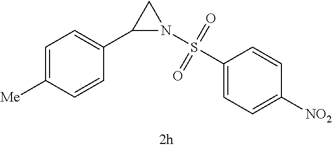 | 89 (83)^{c} |
| 3 | 1e | 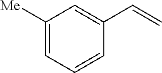 | 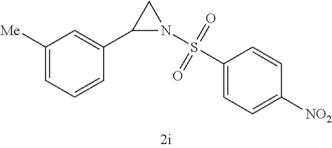 | 89 |
| 4 | 1e | 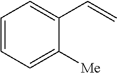 | 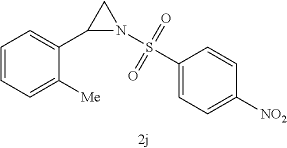 | 88 |
| 5 | 1e | 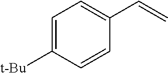 | 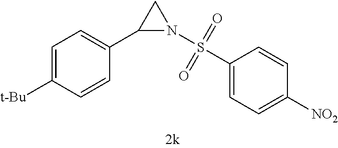 | 98 (97)^{c} |
| 6 | 1e | 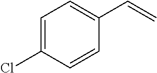 | 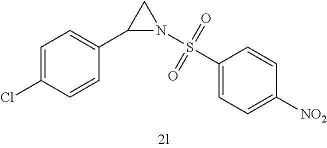 | 94 (83)^{c} |
| 7 | 1e | 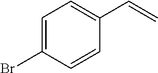 | 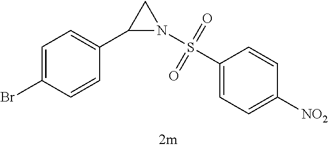 | 96 |
| 8 | 1e | 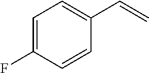 | 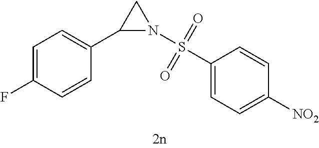 | 95 (90)^{c} |
| 9 | 1e | 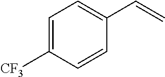 | 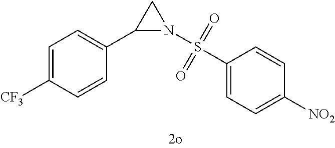 | 96 |
| 10 | 1e | 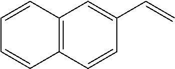 | 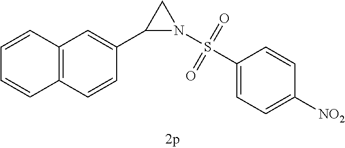 | 75 |
| 11 | 1c | 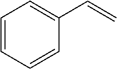 | 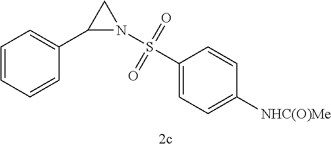 | 98 |
| 12 | 1c | 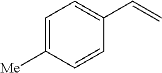 | 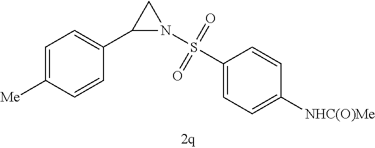 | 83^{d} |
| 13 | 1c | 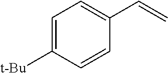 | 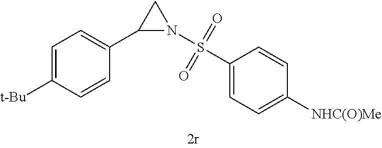 | 84^{d} |
| 14 | 1c | 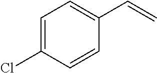 | 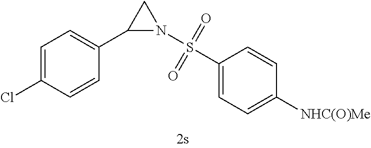 | 93^{d} |

^{a} Reactions were carried out for 18 h in chlorobenzene at 40° C. under N₂ in the presence of 4 Å molecular sieves using 2 mol % [Co(P1)];
Olefin: Azide = 5:1; Concentration:
0.20 mmol azide/1 mL chlorobenzene.
^{b} Isolated yields.
^{c}Olefin: Azide = 1:1.2. Concentration: 0.20 mmol of azide/1 mL of chlorobenzene.
^{d}Performed at 60° C.

General Considerations. All cross-coupling and aziridination reactions were performed under nitrogen in oven-dried glassware following standard Schlenk techniques. 4 Å molecular sieves were dried in a vacuum oven prior to use. Chlorobenzene and dichloromethane were dried over calcium hydride under nitrogen and freshly distilled before use. Toluene and tetrahydrofuran were distilled under nitrogen from sodium benzophenone ketyl prior to use. Acetoamidobenzenesulfonyl azide was purchased from Sigma-Aldrich and used without further purification. Arylsulfonyl chlorides were purchased from commercial sources. Thin layer chromatography was performed on Merck TLC plates (silica gel 60 F254). Flash column chromatography was performed with ICN silica gel (60 Å, 230-400 mesh, 32-63 μm). ¹H NMR and ¹³C NMR were recorded on a Varian Inova400 (400 MHz) or a Varian Inova500 (500 MHz) instrument with chemical shifts reported relative to residual solvent. Infared spectra were measured with a Nicolet Avatar 320 spectrometer with a Smart Miracle accessory. HRMS data was obtained on an Agilent 1100 LC/MS/TOF mass spectrometer.

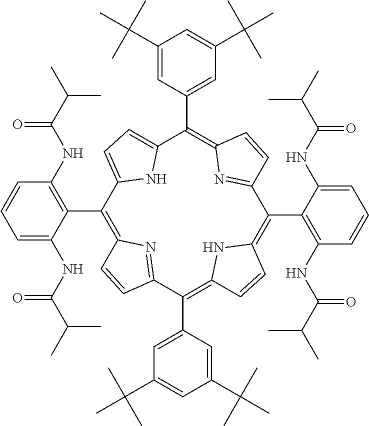

Porphyrin 1 (P1). An oven-dried Schlenk tube equipped with a stirring bar was degassed on vacuum line and purged with nitrogen. The tube was then charged with 5,15-Bis(2,6-dibromophenyl)-10,20-bis[3,5-di(tert-butyl)phenyl]porphyrin (0.2 mmol, 1 eq), isobutylamide (3.2 mmol, 16 eq), Pd(OAc)₂ (0.08 mmol, 40%), Xantphos (0.16 mmol, 80%), Cs₂CO₃ (3.2 mmol, 16 eq). (Chen et al., J. Am. Chem. Soc. 2004, 126, 14718.) The tube was capped with a Teflon screw cap, evacuated and backfilled with nitrogen. After the Teflon screw cap was replaced with a rubber septum, solvent (4-5 mL) was added via syringe. The tube was purged with nitrogen (1-2 min) and the septum was then replaced with the Teflon screw cap and sealed. The reaction mixture was heated in an oil bath at 100° C. with stirring for 72 hours. The resulting reaction mixture was concentrated and the solid residue was purified by flash chromatography (hexanes: ethyl acetate, 7:3) to afford the compound as a purple solid (65-75%, in general). ¹H NMR (400 MHz, CDC1₃): δ8.97 (d, J =4.4 Hz, 4H), 8.85 (d, J=4.8 Hz, 4H), 8.48 (d, J=7.6 Hz, 4H), 8.00 (s, 4H), 7.90-7.85 (m, 4H), 6.46 (s, 4H), 1.52 (s, 36H), 1.20 (m, 4H), 0.31 (d, J=7.4 Hz, 24H), −2.53 (s, 2H). ¹³C NMR (125 MHz, CDC1₃): δ174.7, 149.4, 139.7, 138.8, 133.5, 130.5, 130.1, 123.1, 121.8, 117.8, 108.0, 35.8, 35.0, 31.6, 18.5. UV-vis (CHCl₃), λₘₐₓ, nm (log ε): 425(5.48), 519(4.19), 555(3.84), 595(3.70), 650(3.60). HRMS (ESI): Calcd. for C₇₆H₉₁N₈O₄ ([M+H]⁺) m/z 1179.71578, Found 1179.71870.

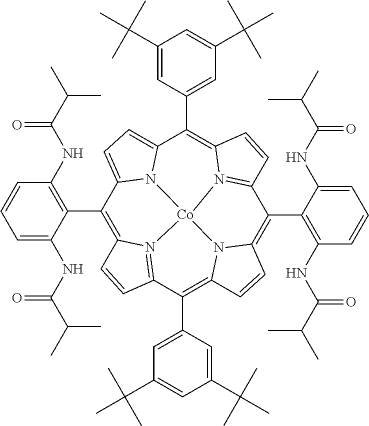

Co[P1] Porphyrin Complex. Porphyrin 1 (0.054 mmol) and anhydrous CoCl₂ (0.43 mmol) were placed in an oven-dried, re-sealable Schlenk tube. The tube was capped with a Teflon screw-cap, evacuated, and backfilled with nitrogen. The screw cap was replaced with a rubber septum, 2,6-lutidine (0.25 mmol) and dry THF (3-4 mL) were added via syringe. The tube was purged with nitrogen for 1-2 minutes, and then the septum was replaced with the Teflon screw cap. The tube was sealed, and its contents were heated in an oil bath at 80° C. with stirring overnight. The resulting mixture was cooled to room temperature, taken up in ethyl acetate, and transferred to a separatory funnel. The mixture was washed with water 3 times and concentrated. The solid residue was purified by flash chromatography (hexanes: ethyl acetate, 6:4) to afford the compound as a purple solid (55.3 mg, 83%). UV-vis (CHCl₃), λₘₐₓ, nm (log E): 415(5.23), 530(4.19). HRMS (ESI): Calcd. for C₇₆H₈₈N₈O₄Co ([M]⁺) m/z 1235.62550, Found 1235.62638.

General Procedure for Synthesis of Azides. (Abramovitch et al., J. Org. Chem. 1977, 42, 2920; Waser et al., J. Am. Chem. Soc. 2006, 128, 11693; Brodsky et al., Org. Lett. 2004, 6, 2619.) A solution of the arylsulfonyl chloride in water: acetone (1:1, 6 ml/mmol) was stirred in a round bottom flask and cooled in an ice bath to 0° C. for 15-20 minutes. Sodium azide (1.5 eq) was added in portions to the sulfonyl chloride mixture and the reaction was monitored by TLC to completion (typically 2-5 hrs). After the reaction was complete, the flask underwent rotary evaporation until the acetone was removed. The crude product was extracted from the water using ethyl acetate or dichloromethane (3×5 ml/mmol). It was then washed with brine (10 ml/mmol), dried over sodium sulfate, and concentrated by rotary evaporation. The resulting oil was then purified by flash column chromatography. The fractions containing product were collected and concentrated by rotary evaporation to afford the compound.

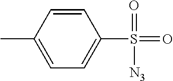

4-Methylbenzenesulfonyl azide (1a, Table 1, entry 1). ¹H NMR (400 MHz, CDC1₃): δ7.82 (d, J=8.4 Hz, 2H), 7.39 (d, J=8.4 Hz, 2H), 2.46 (s, 3H). IR (neat, cm⁻¹): 2123, 1595, 1368, 1162, 1085, 813, 745, 657.

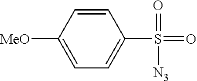

4-methoxybenzenesulfonyl azide (1b, Table 1, entry 2) was obtained using the general procedure as white solid in 97% yield (5.0 g). ¹H NMR (400 MHz, CDC1₃): δ7.89 (d, J=8.4 Hz, 2H), 7.05 (d, J=8.8 Hz, 2H), 3.91 (s, 3H). IR (neat, cm⁻¹): 2126, 1591, 1550, 1518, 1496, 1461, 1442, 1417, 1367, 1265, 1184, 1162, 1109, 1083, 1019, 831, 804, 740, 666.

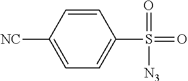

4-Cyanobenzenesulfonyl azide (1d, Table 1, entry 4) was obtained using the general procedure as white solid in 76% yield (592 mg). ¹H NMR (400 MHz, CDC1₃): δ8.08 (d, J=8.4 Hz, 2H), 7.92 (d, J=8.0 Hz, 2H). ¹³C NMR (100 MHz, CDC1₃): δ142.3, 133.4, 128.0, 118.5, 116.6. IR (neat, cm⁻¹): 2238, 2140, 1403, 1366, 1286, 1180, 1158, 1084, 1021, 836, 800, 786, 751, 631.

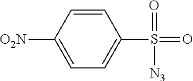

4-Nitrobenzenesulfonyl azide (1e, Table 1, entry 5) was obtained using the general procedure as tan solid in 75% yield (7.76 g). ¹H NMR (400 MHz, CDC1₃): δ8.46 (d, J=8.8 Hz, 2H), 8.17 (d, J=8.8 Hz, 2H). IR (neat, cm⁻¹): 2140, 1605, 1528, 1404, 1374, 1349, 1310, 1175, 1156, 1109, 1084, 1013, 867, 854, 767, 740, 731, 680.

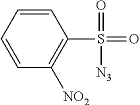

2-Nitrobenzenesulfonyl azide (1f, Table 1, entry 6) was obtained using the general procedure as white solid in 71% yield (7.3 g). ¹H NMR (400 MHz, CDC1₃): δ8.19 (d, J=7.6 Hz, 1H), 7.92-7.80 (m, 3H). IR (neat, cm⁻¹): 2162, 1593, 1552, 1533, 1437, 1366, 1314, 1194, 1171, 1144, 1119, 1056, 966, 853, 782, 755, 736, 730, 694, 650, 605.

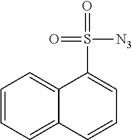

Naphthalene-1-sulfonyl azide (1g, Table 1, entry 7) was obtained using the general procedure as white solid in 94% yield (1.9 g). ¹H NMR (400 MHz, CDC1₃): δ8.57 (d, J=8.8 Hz, 1H), 8.35 (d, J=7.6 Hz, 1H), 8.19 (d, J=8.4 Hz, 1H), 7.99 (d, J=8.0 Hz, 1 H), 7.75 (t, J=8.0 Hz, 1 H), 7.66 (t, J=7.6 Hz, 1H), 7.60 (t, J=8.0 Hz, 1H). ¹³C NMR (100 MHz, CDC1₃): δ136.3, 134.2, 133.4, 130.0, 129.1, 128.0, 127.5, 134.4, 123.9. IR (neat, cm⁻¹): 2131, 1593, 1565, 1505, 1356, 1266, 1194, 1164, 1143, 1134, 1070, 1026, 975, 955, 921, 863, 829, 796, 767, 737, 677, 626.

General Procedure for Aziridination. An oven dried Schlenk tube, that was previously evacuated and backfilled with nitrogen gas, was charged with azide (if solid, 0.2 mmol), catalyst (0.004 mmol), and 4 Å MS (100 mg). The Schlenk tube was then evacuated and back filled with nitrogen. The Teflon screw cap was replaced with a rubber septum and 0.2 ml portion of solvent was added followed by styrene (1.0 mmol), another portion of solvent, then azide (if liquid, 0.2 mmol), and the remaining solvent (total 1 mL). The Schlenk tube was then purged with nitrogen for 1 minute and the rubber septum was replaced with a Teflon screw cap. The Schlenk tube was then placed in an oil bath for the desired time and temperature. Following completion of the reaction, the reaction mixture was purified by flash chromatography. The fractions containing product were collected and concentrated by rotary evaporation to afford the compound.

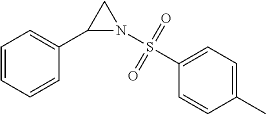

2-Phenyl-1-tosylaziridine (2a, Table 1, entry 1) was obtained using the general procedure as colorless oil in 94% yield (51.4 mg). (Gao et al., Org. Lett. 2005, 7, 3191.) ¹H NMR (400 MHz, CDC1₃): δ7.87 (d, J=8.4 Hz, 2H), 7.33 (d, J=8.4 Hz, 2H), 7.31-7.27 (m, 3H), 7.20 (m, 2H), 3.78 (dd, J=7.2, 4.4 Hz, 1H), 2.98 (d, J=7.2 Hz, 1H), 2.43 (s, 3H), 2.38 (d, J=4.4 Hz, 1H). ¹³C NMR (125 MHz, CDC1₃): δ144.5, 134.97, 134.91, 129.6, 128.4, 128.2, 127.8, 126.4, 40.94, 35.84, 21.55.IR (neat, cm⁻¹): 2923, 2854, 1595, 1495, 1458, 1385, 1319, 1307, 1290, 1232, 1188, 1155, 1134, 1117, 1093, 1082, 907, 815, 799, 780, 754, 711, 696, 687, 662, 634. HRMS (ESI): Calcd. for C₁₅H₁₆NO₂S ([M+H]⁺) m/z 274.08963, Found 274.08987.

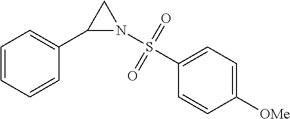

1-(4-Methoxyphenylsulfonyl)-2-phenylaziridine (2b, Table 1, entry 2) was obtained using the general procedure as white solid in 88% yield (51.0 mg). ¹H NMR (400 MHz, CDC1₃): δ7.92 (d, J=8.8 Hz, 2H), 7.28 (m, 3H), 7.21 (m, 2H), 6.99 (d, J=8.8 Hz, 2H), 3.74 (dd, J=7.2, 4.0 Hz, 1 H), 3.87 (s, 3H), 2.96 (d, J=7.2 Hz, 1H), 2.38 (d, J=4.0 Hz, 1H). ¹³C NMR (125 MHz, CDC1₃): δ163.6, 135.0, 130.0, 129.3, 128.4, 128.2, 126.4, 114.2, 55.6, 40.9, 35.8. IR (neat, cm⁻¹): 2958, 2924, 2854, 1592, 1576, 1498, 1458, 1442, 1322, 1301, 1259, 1192, 1150, 1116, 1093, 1017, 908, 836, 805, 779, 755, 721, 691, 667, 629. HRMS (ESI): Calcd. for C₁₅H₁₆NO₃S ([M+H]⁺) m/z 290.08454, Found 290.08488.

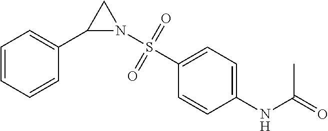

N-(4-(2-Phenylaziridin-1-ylsulfonyl)phenyl)acetamide (2c, Table 1, entry 3) was obtained using the general procedure as tan solid in 98% yield (62.2 mg). ¹H NMR (400 MHz, CDC1₃): δ7.90 (d, J=8.8 Hz, 2H), 7.67 (d, J=8.4 Hz, 2H), 7.61 (bs, 1H), 7.27 (m, 3H), 7.20 (m, 2H), 3.76 (dd, J=7.2, 4.4 Hz, 1H), 2.97 (d, J=7.2 Hz, 1H), 2.39 (d, J=4.4 Hz, 1H), 2.19 (s, 3H). ¹³C NMR (125 MHz, CDC1₃): δ168.8, 142.9, 134.7, 132.0, 129.1, 128.5, 128.4, 126.4, 119.2, 41.1, 36.0, 24.6. IR (neat, cm⁻¹): 3264, 2969, 2924, 1676, 1606, 1587, 1540, 1496, 1400, 1369, 1323, 1265, 1158, 1093, 908, 838, 823, 805, 779, 760, 728, 719, 697, 682, 668, 638, 623. HRMS (ESI): Calcd. for C₁₆H₁₇N₂O₃S ([M+H]⁺) m/z 317.09544, Found 317.09508.

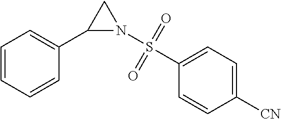

4-(2-Phenylaziridin-1-ylsulfonyl)benzonitrile (2d, Table 1, entry 4) was obtained using the general procedure as a white solid in 89% yield (50.8 mg). ¹H NMR (400 MHz, CDC1₃): δ8.10 (d, J=8.4 Hz, 2H), 7.83 (d, J=8.0 Hz, 2H), 7.30 (m, 3H), 7.21 (m, 2H), 3.88 (dd, J=7.2, 4.8 Hz, 1H), 3.08 (d, J=7.2 Hz, 1H), 2.48 (d, J=4.8 Hz, 1H). ¹³C NMR (100 MHz, CDC1₃): δ206.9, 142.0, 133.5, 132.5 128.3, 128.1, 126.1, 117.0, 116.7, 41.4, 36.2. IR (neat, cm⁻¹): 2233, 1458, 1403, 1333, 1291, 1242, 1187, 1162, 119, 1094, 1020, 973, 909, 844, 797, 758, 749, 724, 699, 682, 644, 624. HRMS (ESI): Calcd. for C₁₅H₁₃N₂O₂S ([M+H]⁺) m/z 285.06922, Found 285.07029.

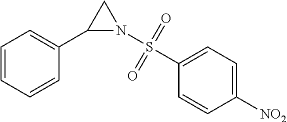

1-(4-Nitrophenylsulfonyl)-2-phenylaziridine (2e, Table 1, entry 5) was obtained using the general procedure as a white solid in 97% yield (58.9 mg). (Mueller et al., Tetrahedron 1996, 52, 1543.) ¹H NMR (400 MHz, CDC1₃): δ8.37 (d, J=8.8 Hz, 2H), 8.19 (d, J=8.8 Hz, 2H), 7.31 (m, 3H), 7.22 (m, 2H), 3.90 (dd, J=7.2, 4.4 Hz, 1H), 3.11 (d, J=7.6 Hz, 1H), 2.50 (d, J=4.4 Hz, 1H). ¹³C NMR (125 MHz, CDC1₃): δ150.6, 143.9, 134.1, 129.1, 128.7, 128.1, 126.4, 124.3, 41.8, 36.5. IR (neat, cm⁻¹): 3110, 2923, 1607, 1527, 1461, 1348, 1307, 1292, 1192, 1157, 1093, 977, 908, 866, 858, 811, 774, 759, 745, 707, 691, 680, 619. HRMS (ESI): Calcd. for C₁₄H₁₃N₂O₄S ([M+H]⁺) m/z 305.05905, Found 305.05901.

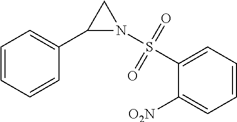

1-(2-Nitrophenylsulfonyl)-2-phenylaziridine (2f, Table 1, entry 6) was obtained using the general procedure as tan oil in 96% yield (58.5 mg). (Kim et al., Angew. Chem., Int. Ed. 2004, 43,-3952.) ¹H NMR (400 MHz, CDC1₃): δ8.23 (d, J=6.4 Hz, 1H), 7.74 (m, 3H), 7.32 (m, 5H), 3.76 (m, 1H), 3.24 (d, J=7.6 Hz, 1H), 2.63 (d, J=4.4 Hz, 1H). ¹³C NMR (125 MHz, CDC1₃): δ148.5, 134.6, 134.4, 132.1, 131.9, 131.2, 128.59, 128.56, 126.5, 124.3, 42.8, 38.0. IR (neat, cm⁻¹): 3094, 2921, 1540, 1461, 1365, 1331, 1192, 1163, 1126, 1066, 1017, 979, 908, 851, 774, 750, 745, 697, 654, 631. HRMS (ESI): Calcd. for C₁₄H₁₃N₂O₄S ([M+H]⁺) m/z 305.05905, Found 305.05928.

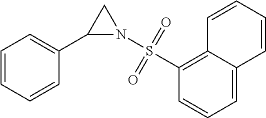

1-(Naphthalen-1-ylsulfonyl)-2-phenylaziridine (2g, Table 1, entry 7) was obtained using the general procedure as white solid in 97% yield (60.0 mg). ¹H NMR (400 MHz, CDC1₃): δ9.00 (d, J=8.4 Hz, 1H), 8.27 (d, J=7.2 Hz, 1H), 8.11 (d, J=8.0 Hz, 1H), 7.93 (d, J=8.0 Hz, 1H), 7.70 (t, J=7.6 Hz, 1H), 7.61 (t, J=7.6 Hz, 1H), 7.55 (t, J=8.0 Hz, 1H), 7.25 (m, 3H), 7.20 (m, 2H), 3.76 (m, 1H), 3.09 (d, J=7.2 Hz, 1H), 2.38 (d, J=4.4 Hz, 1H). ¹³C NMR (125 MHz, CDC1₃): δ135.17, 135.14, 134.1, 133.3, 129.4, 129.0, 128.6, 128.4, 128.27, 128.22, 127.0, 126.4, 125.7, 123.9, 41.1, 36.7. IR (neat, cm⁻¹): 3060, 1594, 1507, 1459, 1384, 1319, 1191, 1161, 1132, 1110, 1083, 1027, 976, 906, 831, 803, 768, 708, 694, 672, 627, 601. HRMS (ESI): Calcd. for C₁₈H₁₆NO₂S ([M+H]⁺) m/z 310.08963, Found 310.08908.

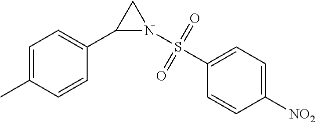

1-(4-Nitrophenylsulfonyl)-2-p-tolylaziridine (2h, Table 2, entry 2) was obtained using the general procedure as tan solid in 89% yield (56.5 mg). ¹H NMR (400 MHz, CDC1₃): δ8.36 (d, J=8.8 Hz, 2H), 8.17 (d, J=8.8 Hz, 2H), 7.10 (m, 4H), 3.86 (dd, J=7.2, 4.8 Hz, 1H), 3.10 (d, J=7.2 Hz, 1H), 2.50 (d, J=4.8 Hz, 1H), 2.31 (s, 3H). ¹³C NMR (125 MHz, CDC1₃): δ150.6, 144.0, 138.6, 131.0, 129.4, 129.1, 126.3, 124.3, 41.9, 36.4, 21.1. IR (neat, cm⁻¹): 3109, 2958, 1606, 1524, 1347, 1322, 1307, 1290, 1157, 1190, 1092, 977, 912, 866, 855, 817, 794, 752, 746, 729, 697, 679, 668, 611. HRMS (ESI): Calcd. for C₁₅H₁₅N₂O₄S ([M+H]⁺) m/z 319.07470, Found 319.07413.

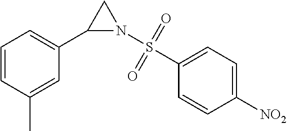

1-(4-Nitrophenylsulfonyl)-2-m-tolylaziridine (2i, Table 2, entry 3) was obtained using the general procedure as tan solid in 89% yield (57.0 mg). (Li et al., J. Org. Chem. 2006, 71, 5876.) ¹H NMR (400 MHz, CDC1₃): δ8.37 (d, J=8.8 Hz, 2H), 8.18 (d, J=8.4 Hz, 2H), 7.20 (t, J=8.0 Hz, 1H), 7.11 (d, J=7.6 Hz, 1H), 7.01 (m, 2H), 3.86 (dd, J=7.2, 4.8 Hz, 1H), 3.09 (d, J=7.2 Hz, 1H), 2.50 (d, J=4.8 Hz, 1H), 2.31 (s, 3H). ¹³C NMR (125 MHz, CDCl₃): δ150.6, 143.9, 138.5, 134.0, 129.5, 129.1, 128.6, 127.0, 124.3, 123.5, 41.9, 36.5, 21.3. IR (neat, cm⁻¹): 3107, 2924, 1607, 1525, 1489, 1457, 1348, 1324, 1307, 1292, 1215, 1156, 1112, 1092, 979, 930, 900, 866, 854, 807, 783, 751, 711, 688, 669, 620. HRMS (ESI): Calcd. for C₁₅H₁₅N₂O₄S ([M+H]⁺) m/z 319.07470, Found 319.07410.

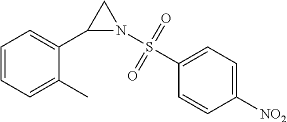

1-(4-Nitrophenylsulfonyl)-2-o-tolylaziridine (2j, Table 2, entry 4) was obtained using the general procedure as tan solid in 88% yield (56.2 mg). ¹H NMR (400 MHz, CDC1₃): δ8.40 (d, J=8.4 Hz, 2H), 8.22 (d, J=8.4 Hz, 2H), 7.23-7.11 (m, 3H), 7.06 (d, J=7.6 Hz, 1H), 3.01 (m, 1H), 3.10 (d, J=7.2 Hz, 1H), 2.43 (d, J=4.8 Hz, 1H), 2.41 (s, 3H). ¹³C NMR (125 MHz, CDC1₃): δ150.6, 143.9, 136.7, 132.3, 130.1, 129.2, 128.4, 126.2, 125.5, 124.3, 40.2, 35.8, 19.0. IR (neat, cm⁻¹): 2980, 1607, 1524, 1349, 1328, 1306, 1243, 1203, 1158, 1092, 1012, 976, 907, 867, 829, 766, 744, 742, 698, 680, 668, 621. HRMS (ESI): Calcd. for C₁₅H₁₅N₂O₄S ([M+H]⁺) m/z 319.07470, Found 319.07415.

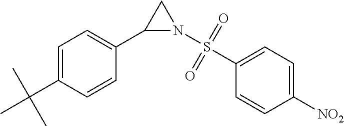

2-(4-tert-Butylphenyl)-1-(4-nitrophenylsulfonyl)aziridine (2k, Table 2, entry 5) was obtained using the general procedure as tan oil in 98% yield (71.0 mg). ¹H NMR (400 MHz, CDC1₃): δ8.37 (d, J=8.4 Hz, 2H), 8.19 (d, J=8.4 Hz, 2H), 7.34 (d, J=8.0 Hz, 2H), 7.15 (d, J=8.0 Hz, 2H), 3.89 (m, 1H), 3.09 (d, J =7.2 Hz, 1H), 2.51 (d, J=4.8 Hz, 1H), 1.28 (s, 9H). ¹³C NMR (125 MHz, CDC1₃): δ151.9, 150.6, 144.0, 131.0, 129.1, 126.1, 125.6, 124.3, 41.9, 36.7, 34.6, 31.2. IR (neat, cm⁻¹): 3060, 2964, 1594, 1533, 1507, 1459, 1320, 1191, 1161, 1133, 1110, 1086, 1027, 977, 907, 832, 804, 770, 744, 696, 673, 628, 604. HRMS (ESI): Calcd. for C₁₈H₂₁N₂O₄S ([M+H]⁺) m/z 361.12165, Found 361.12077.

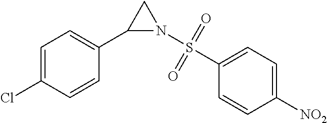

2-(4-Chlorophenyl)-1-(4-nitrophenylsulfonyl)aziridine (2l, Table 2, entry 6) was obtained using the general procedure as white solid in 94% yield (63.5 mg). (Leung et al., J. Am. Chem. Soc. 2005, 127, 16629.) ¹H NMR (400 MHz, CDC1₃): δ8.37 (d, J=8.8 Hz, 2H), 8.17 (d, J=8.8 Hz, 2H), 7.28 (d, J=8.8 Hz, 2H), 7.15 (d, J=8.4 Hz, 2H), 3.87 (dd, J=7.2, 4.8 Hz, 1H), 3.10 (d, J=7.2 Hz, 1H), 2.54 (d, J=4.8 Hz, 1H). ¹³C NMR (125 MHz, CDC1₃): δ150.6, 143.6, 134.6, 132.7, 129.1, 128.9, 127.7, 124.3, 40.9, 36.7. IR (neat, cm⁻¹): 3109, 2958, 2925, 1607, 1523, 1494, 1345, 1323, 1306, 1156, 1091, 1016, 980, 911, 867, 834, 803, 753, 743, 724, 694, 680, 658, 630, 604. HRMS (ESI): Calcd. for C₁₄H₁₂N₂O₄SCl ([M+H]⁺) m/z 339.02008, Found 339.02007.

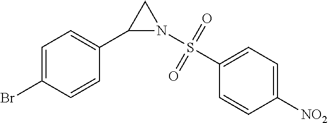

2-(4-Bromophenyl)-1-(4-nitrophenylsulfonyl)aziridine (2m, Table 2, entry 7) was obtained using the general procedure as white solid in 96% yield (73.5 mg). (Ryan et al., Org. Biomol. Chem. 2004, 2, 3566.) ¹H NMR (400 MHz, CDC1₃): δ8.38 (d, J=8.8 Hz, 2H), 8.18 (d, J=8.8 Hz, 2H), 7.20 (m, 2H), 7.00 (t, J=8.4 Hz, 2H), 3.87 (m, 1H), 3.09 (d, J=7.2 Hz, 1H), 2.45 (d, J=4.4 Hz, 1H). ¹³C NMR (125 MHz, CDC1₃): δ150.6, 143.6, 133.2, 131.8, 129.1, 128.0, 124.3, 122.7, 41.1, 36.6. IR (neat, cm⁻¹): 2979, 2924, 1607, 1532, 1491, 1336, 1348, 1319, 1161, 1091, 1009, 981, 906, 854, 802, 769, 753, 739, 722, 688, 668, 625, 617. HRMS (ESI): Calcd. for C₁₄H₁₂N₂O₄SBr ([M+H]⁺) m/z 382.96957, Found 382.96952.

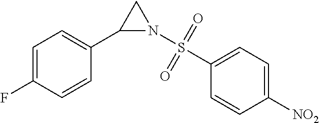

2-(4-Fluorophenyl)-1-(4-nitrophenylsulfonyl)aziridine⁸ (2n, Table 2, Entry 8) was obtained using the general procedure as white solid in 95% yield (61.5 mg). ¹H NMR (400 MHz, CDC1₃): δ8.38 (d, J=8.8 Hz, 2H), 8.18 (d, J=8.8 Hz, 2H), 7.19 (m, 2H), 7.00 (m, 2H), 3.88 (dd, J=7.2, 4.4 Hz, 1H), 3.09 (d, J=7.2 Hz, 1H), 2.47 (d, J=4.4 Hz, 1H). ¹³C NMR (100 MHz, CDC1₃): δ150.9, 143.9, 133.5, 132.1, 129.4, 128.31, 124.6, 123.0, 41.32, 36.92. IR (neat, cm⁻¹): 3109, 1611, 1523, 1512, 1455, 1348, 1323, 1308, 1292, 1231, 1187, 1157, 1120, 1092, 981, 911, 868, 836, 817, 796, 754, 746, 734, 715, 695, 680, 611. HRMS (ESI): Calcd. for C₁₄H₁₂N₂O₄FS ([M+H]⁺) m/z 323.04963, Found 323.04920.

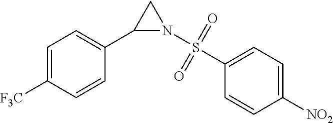

1-(4-Nitrophenylsulfonyl)-2-(4-(trifluoromethyl)phenyl)aziridine⁷ (2o, Table 2, entry 9) was obtained using the general procedure as a white solid in 96% yield (71.8 mg). ¹H NMR (400 MHz, CDC1₃): δ8.39 (d, J=8.4 Hz, 2H), 8.19 (d, J=8.8 Hz, 2H),, 7.58 (d, J=8.4 Hz, 2H), 7.35 (d, J=8.0 Hz, 2H), 3.94 (dd, J=7.2, 4.8 Hz, 1H), 3.14 (d, J=7.2 Hz, 1H), 2.48 (d, J=4.4 Hz, 1H). ¹³C NMR (100 MHz, CDC1₃): δ150.8, 143.6, 138.3, 130.9 (CF₃), 129.2, 126.8, 125.76, 125.73, 124.4, 40.83, 36.87. IR (neat, cm⁻¹): 3112, 2927, 1621, 1608, 1530, 1348, 1322, 1162, 1116, 1091, 1066, 1017, 982, 909, 849, 756, 713, 696, 630. HRMS (ESI): Calcd. for C₁₅H₁₂N₂O₄ F₃S ([M+H]⁺) m/z 373.04644, Found 373.04658.

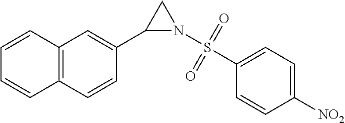

2-(Naphthalen-2-yl)-1-(4-nitrophenylsulfonyl)aziridine (2p, Table 2, entry 10) was obtained using the general procedure as tan solid in 75% yield (53.5 mg). ¹H NMR (400 MHz, CDC1₃): δ8.38 (d, J=8.4 Hz, 2H), 8.21 (d, J=8.8 Hz, 2H), 7.81 (m, 3H), 7.74 (s, 1H), 7.50 (m, 2H), 7.27 (m, 1H), 4.07 (m, 1H), 3.20 (d, J=7.2 Hz, 1H), 2.63 (d, J=4.4 Hz, 1H). ¹³C NMR (125 MHz, CDC1₃): δ150.6, 143.9, 133.2, 132.9, 131.4, 129.1, 128.7, 127.75, 127.73, 126.6, 126.5, 126.2, 124.3, 123.2, 42.2, 36.6. IR (neat, cm⁻¹): 3107, 2922, 1604, 1529, 1401, 1346, 1326, 1305, 1156, 1092, 949, 917, 862, 852, 800, 767, 742, 713, 679, 669, 640, 623, 608. HRMS (ESI): Calcd. for C₁₈H₁₅N₂O₄S ([M+H]⁺) m/z 355.07470, Found 355.07456.

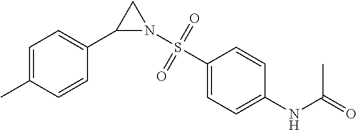

N-(4-(2-p-Tolylaziridin-1-ylsulfonyl)phenyl)acetamide (2q, Table 2, entry 12) was obtained using the general procedure as tan oil in 83% yield (55.1 mg). ¹H NMR (400 MHz, CDC1₃): δ7.98 (s, 1H), 7.86 (d, J=8.8 Hz, 2H), 7.67 (d, J=8.8 Hz, 2H), 7.08 (s, 4H), 3.71 (dd, J=6.8, 4.4 Hz, 1H), 2.93 (d, J=6.8 Hz, 1H), 2.39 (d, J=4.4 Hz, 1H), 2.29 (s, 3H), 2.16 (s, 3H). ¹³C NMR (100 MHz, CDC1₃): δ168.9, 148.9, 138.3, 132.0, 131,6, 129.2, 129.1, 126.3, 119.2, 41.2, 35.8, 24.6, 21.2. IR (neat, cm⁻¹): 3346, 3111, 1701, 1590, 1529, 1402, 1370, 1320, 1261, 1155, 1093, 909, 820, 731, 683, 635, 619. HRMS (APCI). Calcd. for C₁₇H₁₉N₂O₃S ([M+H]⁺) m/z 331.11109, Found 331.11052.

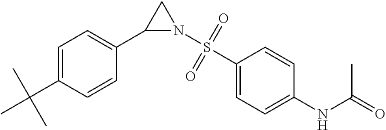

N-(4-(2-(4-tert-Butylphenyl)aziridin-1-ylsulfonyl)phenyl)acetamide (2r, Table 2, entry 13) was obtained using the general procedure as tan oil in 84% yield (62.3 mg). ¹H NMR (400 MHz, CDC1₃): δ8.00 (s, 1H), 7.87 (d, J=8.8 Hz, 2H), 7.68 (d, J=8.8 Hz, 2H), 7.31 (d, J=8.0 Hz, 2H), 7.12 (d, J=8.4 Hz, 2H), 3.74 (dd, J=7.2, 4.8 Hz, 1H), 2.94 (d, J=7.2 Hz, 1H), 2.39 (d, J=4.8 Hz, 1H), 2.17 (s, 3H), 1.27 (s, 9H). ¹³C NMR (100 MHz, CDC1₃): δ169.0, 151.5, 143.0, 132.0, 131.6, 129.1, 126.2, 125.5, 119.2, 41.1, 35.9, 34.5, 31.2, 24.5. IR (neat, cm⁻¹): 3334, 2965, 1703, 1591, 1529, 1402, 1365, 1321, 1263, 1156, 1093, 910, 839, 749, 731, 689, 639, 617. HRMS (APCI): Calcd. for C₂₀H₂₅N₂O₃S ([M+H]⁺) m/z 373.15804, Found 373.15904.

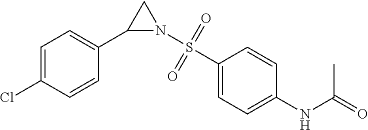

N-(4-(2-(4-Chlorophenyl)aziridin-1-ylsulfonyl)phenyl)acetamide (2s, Table 2, entry 14) was obtained using the general procedure as tan oil in 93% yield (65.3 mg). ¹H NMR (400 MHz, CDC1₃): δ7.98 (s, 1H), 7.84 (d, J=8.8 Hz, 2H), 7.67 (d, J=8.8 Hz, 2H), 7.22 (d, J=8.4 Hz, 2H), 7.10 (d, J=8.4 Hz, 2H), 3.68 (dd, J=7.2, 4.4 Hz, 1H), 2.93 (d, J 32 7.2 Hz, 1H), 2.33 (d, J=4.4 Hz, 1H), 2.16 (s, 3H). ¹³C NMR (100 MHz, CDC1₃): δ168.9, 143.1, 134.2, 133.3, 131.8, 129.1, 128.7, 127.8, 119.2, 40.3, 36.1, 24.7. IR (neat, cm⁻¹): 3333, 3112, 1701, 1590, 1529, 1494, 1402, 1370, 1322, 1262, 1156, 1092, 1014, 981, 908, 827, 776, 735, 689, 637, 623, 613. HRMS (APCI): Calcd. for C₁₆H₁₆N₂O₃SCI ([M+H]⁺) m/z 351.05647, Found 351.05748.

TABLE 3
Asymmetric Olefin Aziridination
with Azides by Chiral Cobalt Porphyins

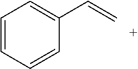

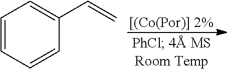

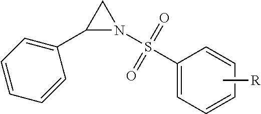

| Entry | R | Co[Por] | Yield (%) | % ee |
|---|---|---|---|---|
| 1 | p-NO₂ | 1 | 94 | 13 |
| 2 | p-NO₂ | 2 | 44 | 81 |
| 3 | p-NO₂ | 3 | 28 | −83 |
| 4 | p-NO₂ | 1 | 96 | 28 |
| 5 | p-NO₂ | 2 | 18 | 87 |
| 6 | p-NO₂ | 3 | 11 | −88 |

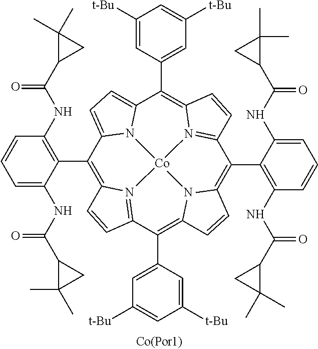

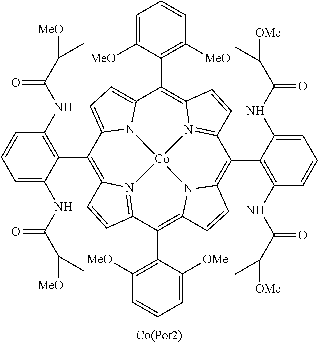

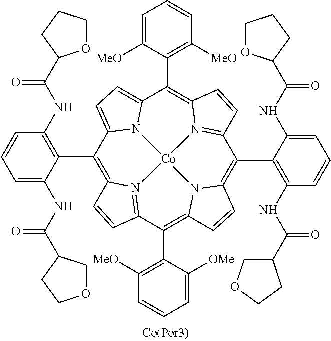

What we claim is:

1. A process for the aziridination of an olefin, the process comprising treating the olefin with a sulfonyl azide in the presence of a metal porphyrin complex.

2. The process of claim 1 wherein the olefin corresponds to Formula 1: 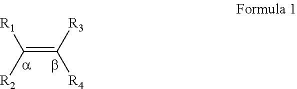
wherein R₁, R₂, R₃, and R₄ are independently hydrogen, hydrocarbyl, substituted hydrocarbyl, heterocyclo or electron withdrawing group.

3. The process of claim 1 wherein the olefin corresponds to Formula 5: 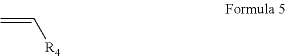
wherein R₄ is hydrogen, hydrocarbyl, substituted hydrocarbyl, or heterocyclo.

4. The process of claim 3 wherein R₄ is aryl.

5. The process of claim 3 wherein R₄ is phenyl or substituted phenyl:

6. The process of claim 5 wherein the sulfonyl azide corresponds to Formula A: 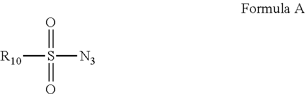
R₁₀ is hydrogen, hydrocarbyl, substituted hydrocarbyl, or heterocyclo.

7. The process of claim 6 wherein R₁₀ is aryl.

8. The process of claim 6 wherein R₁₀ is phenyl or substituted phenyl and the phenyl substituents are selected from the group consisting of alkyl, alkoxy, cyano, and amido.

9. The process of claim 1 wherein the sulfonyl azide is an arylsulfonyl azide.

10. The process of claim 1 wherein the sulfonyl azide corresponds to Formula A: 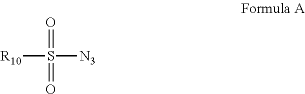
R₁₀ is hydrogen, hydrocarbyl, substituted hydrocarbyl, or heterocyclo.

11. The process of claim 10 wherein R₁₀ is aryl.

12. The process of claim 10 wherein R₁₀ is phenyl or substituted phenyl and the phenyl substituents are selected from the group consisting of alkyl, alkoxy, cyano, and amido.

13. The process of claim 10 wherein R₁₀ is phenyl, tolyl, methoxyphenyl, ethanoylamine phenyl, cyanophenyl, nitrophenyl, or naphthyl.

14. The process of claim 13 wherein the sulfonyl azide is selected from the group consisting of 4-methylbenzenesulfonyl azide, 4-methoxybenzenesulfonyl azide, 4-cyanobenzenesulfonyl azide, 4-nitrobenzenesulfonyl azide, 2-nitrobenzenesulfonyl azide, and naphthalene-1-sulfonyl azide.

15. The process of claim 11 wherein the olefin corresponds to Formula 5: 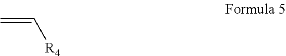
and R₄ is aryl.

16. The process of claim 15 wherein R₄ is phenyl or substituted phenyl.

17. The process of claim 1 wherein the metal porphyrin complex is a cobalt porphyrin complex.

18. The process of claim 5 wherein the metal porphyrin complex is a cobalt porphyrin complex.

19. The process of claim 15 wherein the metal porphyrin has the structure:

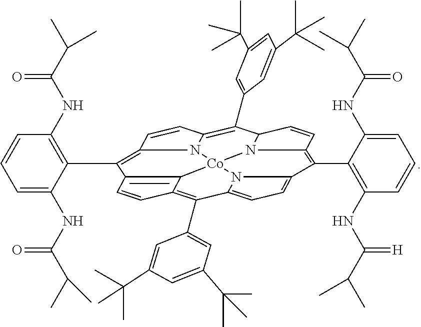

20. A cobalt porphyrin complex having the structure [chemical structure]
